(19) United States
(12) Patent Application Publication
(10) Pub. No.: US 2019/0285634 A1
(43) Pub. Date: Sep. 19, 2019

(54) ANALYSIS OF RESPONSE TO THERAPEUTICS IN CANCER

(71) Applicant: The Board of Trustees of the Leland Stanford Junior University, Stanford, CA (US)

(72) Inventors: Arvin Gouw, San Francisco, CA (US); Alice Fan, Mountain View, CA (US); Dean W. Felsher, San Mateo, CA (US)

(21) Appl. No.: 16/356,582

(22) Filed: Mar. 18, 2019

Related U.S. Application Data
(60) Provisional application No. 62/740,013, filed on Oct. 2, 2018.
(60) Provisional application No. 62/644,303, filed on Mar. 16, 2018.

(51) Int. Cl. G01N 33/574; C12Q 1/02

(52) U.S. Cl. CPC G01N 33/5748; B82Y 15/00; C12Q 1/025

(57) ABSTRACT

A nanoimmunoassay (NIA) is applied to quantify analytes, including without limitation proteins and isoforms of proteins involved in oncogenic or metabolic signaling pathways, in a small amount of lysate from a tissue sample. Samples of interest for NIA include without limitation blood or solid tumor microbiopsy samples, such as fine needle aspirate (FNA) or circulating tumor cells. Samples may be taken at a single timepoint, or may be taken at multiple timepoints. Samples may be as small as 100,000 cells, as small as 5000 cells, as small as 1000 cells, as small as 100 cells, as small as 50 cells, as small as 25 cells or less. The NIA detection method combines size separation of proteins or isoelectric protein focusing and antibody detection in a microfluidic system.

### CROSS REFERENCE

This application claims benefit of U.S. Provisional Patent Application No. 62/740,013, filed Oct. 2, 2018 and U.S. Provisional Patent Application No. 62/644,303, filed Mar. 16, 2018, which applications are incorporated herein by reference in their entirety.

### FEDERALLY SPONSORED RESEARCH AND DEVELOPMENT

This invention was made with Government support under contract CA196585 awarded by the National Institutes of Health. The Government has certain rights in the invention.

### BACKGROUND

Transformation and growth of tumor cells is a complex process, which can be variable even within a particular tissue type. Analytical methods that can define the phenotype of tumor cells are useful in determining appropriate therapy, and are therefore of clinical interest. Additionally, knowledge of the mechanism by which a cancer therapy acts is useful determining optimal formulation and dosage of such agents; in screening for agents effective in treating cancer; and in following patients through a course of treatment.

A variety of changes in protein expression and post translational modification take place during oncogenesis and in response to therapy, for example, reversible phosphorylation, which can be a molecular mechanism by which intracellular signals are transmitted. A substantial number of signaling proteins are kinases or phosphatases that act on serine, threonine, and tyrosine residues. With over 2000 human genes predicted to code for kinases and the potential for each kinase to act on multiple targets, signaling networks are immensely complex. An important step towards unraveling this complexity is the development of new proteomics technologies that can quantitatively monitor the phosphorylation states of signaling proteins in a multiplex fashion. Such technologies enable the detailed analysis of signaling pathways in a global perspective and the rapid identification of previously unrecognized signaling events that can occur in response to therapeutic agents.

Sensitive methods in protein detection that can track changes in proteins, particularly protein isoforms, in response to therapeutic agents are of great clinical interest. The present invention addresses this need.

### SUMMARY

In certain embodiments, a nanofluidic proteomic immunoassay (NIA) is applied to quantify analytes, including without limitation analytes such as protein quantity, protein isoforms, protein phosphoisoforms involved in oncogenic signaling pathways, in a small amount of lysate from a tissue sample. Samples of interest for NIA include without limitation blood and derivatives thereof, such as plasma or cells from the blood, or solid tumor microbiopsy samples, such as fine needle aspirate (FNA) or circulating tumor cells. Samples may be taken at a single timepoint, or may be taken at multiple timepoints. Samples may be as small as 100,000 cells, as small as 5000 cells, as small as 1000 cells, as small as 100 cells, as small as 50 cells, as small as 25 cells or less. The NIA detection method combines size or isoelectric protein focusing and antibody detection in a nanofluidic system. Blood cells may be retained in the sample to reduce variability. Because NIA only need minimal amounts of specimen, the analysis is minimally invasive, allowing for example serial protein profiles to be obtained before and after initiating treatment, allowing the determination of predictive protein biomarkers by quantifying early changes in protein activity in patients starting treatment; and the like.

In some embodiments, the clinical sample is obtained by fine needle aspiration. In some embodiments, the clinical sample is a fine needle aspirate (FNA) of a solid tumor that is sampled in vivo, or cultured ex vivo. In some embodiments, the method further comprises comparing the FNA with an adjacent non-tumor tissue. In some embodiments, the subject is human.

In some embodiments of the invention, the NIA detection is performed on a sample that has been maintained at a temperature of up to about 25° C., e.g. between about 4° C. to about 25° C. An important concern for the stability of phosphorylated proteins, e.g. ERK phosphorylation in samples, such as clinical fine needle aspirate (FNA) specimens, is the time interval that elapses between FNA harvesting and flash-freezing the FNA in the laboratory, e.g. at a temperature of −80° or less, which interval may be referred to as the FNA storage time. The FNA storage time may be up to 12 hours, up to 18 hours, up to 24 hours, up to 36 hours, up to 48 hours, or more; and may be at least 4 hours, at least 6 hours, at least 8 hours, at least 12 hours. The FNA storage may be in cold medium, e.g., RPMI, DME, PBS, etc., from about 4° C. to about 10° C., usually around 4° C. To address these concerns, we performed benchmarking experiments to validate the stability of protein phosphorylation in FNAs from patients with kidney cancer. The relative abundances of phosphorylated and non-phosphorylated isoforms of ERK were shown to be remarkably stable, showing that measurements are not significantly altered by the method of FNA processing, even with a storage time up to 2 days in length. In some embodiments, the clinical sample is flash frozen after the FNA storage time.

Proteins for analysis include, without limitation, KGA and GAC isoforms of glutaminase 1 (GLS1); peroxiredoxin-6 (PRDX6); human carbonic anhydrase 9, human alpha-tubulin; human cyclin D1; human p21; human p27; human retinoblastoma protein (pRb); human receptor tyrosine kinase AXL; human vascular endothelial growth factor receptor 2 (VEGFR2); human PAX8; human PDL1. Analysis may directed to the level of expression, and/or of the distribution of isoforms.

In some embodiments NIA is used to monitor the response of a cancer cell sample, e.g. a patient sample, to therapy. In some embodiments the therapy is radiation therapy. In some embodiments the therapy is a targeted therapy, e.g. a kinase inhibitor. Specific drugs exemplified herein include, for example, cabozantinib, axitinib, trametinib, rigosertib, IQGAP1 WW domain peptide, etc. Radiation and/or immunotherapy may be combined with any of the systemic therapies. The sample is analyzed by NIA following treatment to determine the abundance and distribution of proteins of interest, including isoforms of interest. In particular it is shown that drug treatment results in differential distribution detectable by NIA of protein isoforms including ERK, AKT1, AKT3, MEK2, PRDX6, p70S6K1, S6, PCNA, cyclin D1, cleaved PARP-1, which have a distinct distribution pattern between treated and untreated samples.

In some embodiments a patient is treated with a drug and the biopsy sample obtained following treatment. In other embodiments a biopsy sample is treated ex vivo to determine the responsiveness of the cancer to a therapy of interest, where a treatment may then administered to the patient if the individual sample is demonstrated to be responsive.

### DESCRIPTION OF THE DRAWINGS

FIG. 1A-1B. FIG. 1A: Relative abundance of ERK2 phospho-isoforms (measured by charge-separation NIA using anti-ERK2 antibody [EMD Millipore, Cat#06-182] at 1:300 dilution, average of two technical replicates) in two human RCC tumors (T1 and T2) and one normal kidney (N), comparing FNA processing on the same day (within 1 hour of tissue harvesting, ‘day 0’) vs. 24 hours later (‘day 1’). FIG. 1B: Relative abundance of ERK2 phospho-isoforms (measured by NIA, average of two technical replicates) in two regions (T1 and T2) from the same human RCC tumor and adjacent normal kidney (N), comparing FNA processing on the same day of tissue harvesting (within 1 hour of tissue harvesting, ‘day 0’) vs. 2 days later (40 hours after harvesting, ‘day 2’).

FIG. 2. Relative abundance of ERK2 phospho-isoforms, measured by charge-separation NIA using anti-ERK2 antibody [EMD Millipore, Cat#06-182] at 1:300 dilution, in two regions of the same human RCC tumor (region T1, left panel; region T2, right panel), comparing FNA transport and processing at room temperature vs. on ice. FNAs were processed within 1 hour after harvesting.

FIG. 3. Fine needle aspirates of two regions of the same human RCC tumor (T1 and T2) and adjacent kidney tissue (N) were obtained from 4 patients with kidney cancer. Charge-separation NIA was used to measure levels of both the KGA and GAC isoforms of glutaminase 1 (GLS1) in these samples using anti-GLS1 antibody (Epitomics, Cat# T3472). Left: example of NIA glutaminase trace from an FNA. Right: graphed data for 4 patients, each with 2 FNAs from the tumor (T1, T2), and one FNA from adjacent non-tumor tissue (N).

FIG. 4. Circulating tumor cells were isolated from 10 patients using Mag-Sifter technology: cells from each patient were frozen in pellets. Cell pellets from each patient were analyzed using charge-separation NIA to measure Peroxiredoxin 6 (PRDX6) isoforms with anti-PRDX6 antibody (Abcam, Cat#73350). Patients either had EGFR mutations (MUT), wild type (WT), or unknown EGFR status (A,B,C,D).

FIG. 5A-5I. FIG. 5A Nano-immunoassay (NIA) by size-separation of human carbonic anhydrase 9 (CA9) and human alpha-tubulin (Tubulin). Human kidney cancer cell line 786-0 deficient of the von Hippel-Lindau tumor suppressor (VHL) (786-0, positive control) or reconstituted with VHL (786-0 VHL, negative control) were probed with anti-CA9 (GeneTex, clone GT12, Cat# GTX70020) or anti-tubulin antibody (EMD Millipore, clone DM1A, Cat# MABT205) at indicated dilutions after NIA size separation of whole cell protein lysates. Expected molecular weights: CA9: 55-60 kDa; Tubulin: 55 kDa. FIG. 5B Detection of tubulin by NIA size separation of protein lysates from tissue slice cultures (TSCs) from human kidney cancer tissue expanded in mice as patient-derived xenograft. Slices of tumor tissue from PDX mice were cultured in vitro and treated with either IQGAP1 WW domain peptide (WW), scrambled peptide (scr.; negative control for WW), trametinib (MEK inhibitor) or vehicle (DMSO; negative control for trametinib) for 2 days. Tissue protein lysate was probed with an anti-tubulin antibody (EMD Millipore, clone DM1A, Cat# MABT205, 1:100 dilution). K562: cell line control, untreated of treated with imatinib (+ imat.) at 10 μM for 29 h. FIG. 5C Nano-immunoassay (NIA) by size separation of human Cyclin D1. Human hTERT RPE-1 whole cell lysate at the indicated protein concentration was probed with anti-Cyclin D1 antibody (Thermo Scientific, clone SP4, Cat# RM-9104-S1, 1:50 dilution). Left panel: chemiluminescence signal from NIA plotted against protein concentration to demonstrate linearity of the assay. R²: correlation coefficient. Right panel: Gel view of NIA signal. Expected molecular weight of Cyclin D1: 36 kDa. FIG. 5D Nano-immunoassay (NIA) by size separation of human p21. Human hTERT RPE-1 whole cell lysate at the indicated protein concentration was probed with anti-p21 antibody (Cell Signaling Technology, clone 12D1, Cat#2947, 1:200 dilution). Left panel: chemiluminescence signal from NIA plotted against protein concentration to demonstrate linearity of the assay. R²: correlation coefficient. Right panel: Gel view of NIA signal. Expected molecular weight of p21: 21 kDa. FIG. 5E Nano-immunoassay (NIA) by size separation of human p27. Human hTERT RPE-1 whole cell lysate (2dSS: prepared after 2 days of serum withdrawal from culture medium) at the indicated protein concentration was probed with anti-p27 antibody (Cell Signaling Technology, clone 69C12, Cat#3686, 1:100 dilution). Left panel: chemiluminescence signal from NIA plotted against protein concentration to demonstrate linearity of the assay. R²: correlation coefficient. Right panel: Gel view of NIA signal. Expected molecular weight of p27: 27 kDa. FIG. 5F Nano-immunoassay (NIA) by size separation of human Retinoblastoma protein (pRb). Human hTERT RPE-1 whole cell lysate at the indicated protein concentration was probed with anti-pRb antibody (Cell Signaling Technology, clone D20, Cat#9313, 1:50 dilution). Left panel: chemiluminescence signal from NIA plotted against protein concentration to demonstrate linearity of the assay. R²: correlation coefficient. Right panel: Gel view of NIA signal. Expected molecular weight of pRb: 110 kDa. FIG. 5G Nano-immunoassay (NIA) by size separation of human receptor tyrosine kinase AXL. Human kidney cancer cell line SN12L1 whole cell lysate was probed with anti-AXL antibody. Expected molecular weight of AXL: 138 kDa and higher molecular weight forms. FIG. 5H Nano-immunoassay (NIA) by size separation of human vascular endothelial growth factor receptor 2 (VEGFR2). Human kidney cancer cell line 786-0 whole cell lysate at the indicated protein concentration was probed with anti-VEGFR2 antibody (Cell Signaling Technology, clone 55611, Cat#2479, 1:100 dilution). Left panel: chemiluminescence signal from NIA plotted against protein concentration to demonstrate linearity of the assay. R²: correlation coefficient. Right panel: Gel view of NIA signal. Expected molecular weight of VEGFR2: 210 and 230 kDa. FIG. 5I Nano-immunoassay (NIA) by size separation of human PAX8. Human kidney cancer cell line 786-0 (positive control) and human cell line HEK293 (negative control) whole cell lysate at the indicated protein concentration were probed with three different anti-PAX8 antibodies (P12: Novus Biologicals, Cat# NBP1-32440; P13: GeneTex, Cat# GTX101583; P14: Abcam, clone EPR18715, Cat# ab191870). Expected molecular weight of PAX8: 48 kDa.

FIG. 6A-6B. FIG. 6A Quantification of relative abundance of ERK1 and ERK2 phospho-isoforms in human 786-0 cells treated with cabozantinib (1 μM) or DMSO (negative control) for 1 day and stimulated with VEGF or HGF (10 ng/mL) for 10 min before cell lysis, protein separation by charge using NIA and probing with anti-pan-ERK antibody (EMD Millipore, Cat#06-182, 1:300 dilution). Error bars: standard deviation (SD) of technical triplicates. FIG. 6B Same data as in previous slide, but without the predominant unphosphorylated ERK isoforms. Red boxes: most prominent differences in the abundance of specific ERK phospho-isoforms between cabozantinib- and control-treated cells under stimulation with HGF.

FIG. 7A-7E. FIG. 7A NIA charge separation of tissue protein lysate from human kidney cancer and human normal kidney, probed with anti-AKT2 antibody (Cell Signaling Technology, clone D6G4, Cat#3063). Differently charged isoforms of AKT2 are identified, likely representing unphosphorylated, mono- and multiple-phosphorylated AKT2. FIG. 7B. NIA charge separation of tissue protein lysate from human kidney cancer and human normal kidney, probed with anti-MEK2 antibody (Cell Signaling Technology, Cat#9125, 1:50 dilution). Differently charged isoforms of MEK2 are identified, likely representing unphosphorylated, and phosphorylated MEK2. FIG. 7C NIA charge separation of tissue protein lysate from human kidney cancer, biopsied in vivo during surgery and again ex vivo after surgical tumor removal, and probed with anti-pan-ERK antibody (EMD Millipore, Cat#06-182, 1:300 dilution). The overall ERK phosphorylation profile is similar in both tissue samples with unphosphorylated ERK2 (ERK2) and mono-phosphorylated ERK2 (pERK2) being the dominant ERK isoforms. FIG. 7D NIA charge separation of whole cell lysate from cells isolated from blood from a human kidney cancer patient. Cells were isolated from blood drawn on the day of surgical kidney tumor removal and again from blood drawn on day 1 after surgery. Cells were cultured and expanded on VitaAssay plates and lysate was probed with an anti-pan-ERK antibody (EMD Millipore, Cat#06-182, 1:300 dilution). FIG. 7E NIA charge separation of whole cell lysate from cells isolated from blood from a human non-small cell lung cancer patient. Cells were isolated from blood drawn on the day of start of radiation treatment and again from blood drawn on day 3 of radiation treatment. Cells were cultured and expanded on VitaAssay plates and lysate was probed with an anti-PRDX6 antibody (Abcam, Cat#73350). Multiple PRDX6 isoforms of different charges are identified and profiles differ pre- and post-treatment.

FIG. 8A-8H. FIG. 8A NIA charge separation of whole cell lysate from human lymphoma tissue expanded in mice as patient-derived xenograft and treated with either cabozantinib or vehicle (negative control) for 2 days. Tissue protein lysate was probed with an anti-pan-ERK antibody (EMD Millipore, Cat# ABS44, 1:50 dilution). FIG. 8B NIA charge separation of whole cell lysate from human lymphoma tissue expanded in mice as patient-derived xenograft and treated with either cabozantinib or vehicle (negative control) for 2 days. Tissue protein lysate was probed with an anti-AKT3 antibody (EMD Millipore, Cat#07-383, 1:50 dilution). FIG. 8C NIA charge separation of whole cell lysate from human lymphoma tissue expanded in mice as patient-derived xenograft. Tissue protein lysate was probed with antibodies specific to AKT1 (R&D Systems, Cat# AF1775, 1:50 dilution), AKT2 (Cell Signaling Technology, clone D6G4, Cat#3063, 1:50 dilution), and AKT3 (EMD Millipore, Cat#07-383, 1:50 dilution), identifying differentially charged (likely phosphorylated) isoforms of each AKT isoform. FIG. 8D NIA charge separation of whole cell lysate from human lymphoma tissue expanded in mice as patient-derived xenograft. Tissue protein lysate was probed with anti-p70S6K1 antibody (Santa Cruz Biotechnologies, clone H-8, Cat# sc-8418, 1:50 dilution) identifying two differently charged isoforms of p70 S6 Kinase 1, likely representing different phosphorylation status of the protein. FIG. 8E NIA charge separation of whole cell lysate from human lymphoma tissue expanded in mice as patient-derived xenograft. Tissue protein lysate was probed with anti-phospho S6 antibody (Cell Signaling Technology, clone D68F8, Cat#5364, 1:50 dilution) identifying three differently charged isoforms of phosphorylated S6 protein. FIG. 8F Relative ERK phospho-isoform abundance quantified by NIA charge assay. Human lymphoma was expanded in mouse as patient-derived xenograft (CDX 1^{st} gen) and passaged to a cohort of second-generation PDX mice (CDX 2^{nd} gen). Second-generation PDX mice were then treated with either cabozantinib, axitinib, of vehicle (negative control) for 2 days, then tissue was harvested, and protein lysate was separated by NIA charge assay and probed with an anti-pan-ERK antibody (EMD Millipore, Cat# ABS44, 1:50 dilution). The relative abundance for each phospho-isoform of ERK1 and ERK2 was quantified as fraction of total ERK1 or ERK2, respectively. Error bars: Standard error of the mean across three technical replicates. FIG. 8G Phospho-ERK expression levels quantified by NIA charge assay. Quantitative ERK measurements (‘normalized area’) of the phosphorylated isoforms of ERK1 and ERK2 in human lymphoma expanded as patient-derived xenograft in mice (same samples as in previous slide) were normalized to HSP70 (‘housekeeping’ gene, antibody: Santa Cruz Biotechnology, clone W27, Cat# sc-24, 1:500 dilution). Error bars: Standard error of the mean across three technical replicates. FIG. 8H Total ERK expression levels quantified by NIA charge assay. Quantitative ERK measurements (‘normalized area’) of the total amount of ERK1 and ERK2 in human lymphoma expanded as patient-derived xenograft in mice (same samples as in previous slide) were normalized to HSP70 (‘housekeeping’ gene, antibody: Santa Cruz Biotechnology, clone W27, Cat# sc-24, 1:500 dilution). Error bars: Standard error of the mean across three technical replicates.

FIG. 9A-9G. FIG. 9A. Detection of cleaved PARP-1 (apoptosis marker) by NIA size separation of protein lysates from tissue slice cultures (TSCs) from human kidney cancer tissue expanded in mice as patient-derived xenograft. Slices of tumor tissue from PDX mice were cultured in vitro and treated with either IQGAP1 WW domain peptide (WW), scrambled peptide (scr.; negative control for WW), trametinib (MEK inhibitor) or vehicle (DMSO; negative control for trametinib) for 2 days. Tissue protein lysate was probed with an anti-cl. PARP-1 antibody (EMD Millipore, Cat# ABC26, 1:100 dilution). K562: cell line control, untreated (negative control) or treated with imatinib (+ imat.; positive control) at 10 μM for 29 h. FIG. 9B Detection of PCNA (proliferating cell nuclear antigen; marker of proliferating cells in the S/G2 phases of the cell cycle) by NIA size separation of protein lysates from tissue slice cultures (TSCs) from human kidney cancer tissue expanded in mice as patient-derived xenograft. Slices of tumor tissue from PDX mice were cultured in vitro and treated with either IQGAP1 WW domain peptide (WW), scrambled peptide (scr.; negative control for WW), trametinib (MEK inhibitor) or vehicle (DMSO; negative control for trametinib) for 2 days. Tissue protein lysate was probed with an anti-PCNA antibody (Cell Signaling Technology, clone PC10, Cat#2586, 1:50 dilution). K562: cell line control, untreated (positive control) or treated with imatinib (+ imat.; negative control) at 10 μM for 29 h. FIG. 9C Detection of Cyclin D1 (marker of proliferating cells in the G1 phase of the cell cycle) by NIA size separation of protein lysates from tissue slice cultures (TSCs) from human kidney cancer tissue expanded in mice as patient-derived xenograft. Slices of tumor tissue from PDX mice were cultured in vitro and treated with either IQGAP1 WW domain peptide (WW), scrambled peptide (scr.; negative control for WW), trametinib (MEK inhibitor) or vehicle (DMSO; negative control for trametinib) for 2 days. Tissue protein lysate was probed with an anti-Cyclin D1 antibody (Thermo Scientific, clone SP4, Cat# RM-9104-S0). K562: cell line control, untreated or treated with imatinib (+ imat.) at 10 μM for 29 h. FIG. 9D Expression levels of ERK phospho-isoforms quantified by NIA charge assay in tissue slice cultures (TSCs) from human kidney cancer tissue expanded in mice as patient-derived xenograft. Slices of tumor tissue from PDX mice were cultured in vitro and treated with either IQGAP1 WW domain peptide (WW), scrambled peptide (scr.; negative control for WW), trametinib (MEK inhibitor) or vehicle (DMSO; negative control for trametinib) for 2 days. Tissue protein lysate was probed with an anti-pan-ERK antibody (EMD Millipore, Cat# ABS44, 1:50 dilution). The expression level of each phospho-isoform of ERK1 and ERK2 was quantified by normalization to HSP70 (‘housekeeping’ gene, antibody: Santa Cruz Biotechnology, clone W27, Cat# sc-24, 1:500 dilution). Error bars: Standard error of the mean across three technical replicates. FIG. 9E Relative abundance of ERK phospho-isoforms quantified by NIA charge assay in tissue slice cultures (TSCs) from human kidney cancer tissue expanded in mice as patient-derived xenograft. Slices of tumor tissue from PDX mice were cultured in vitro and treated with either IQGAP1 WW domain peptide (WW), scrambled peptide (scr.; negative control for WW), trametinib (MEK inhibitor) or vehicle (DMSO; negative control for trametinib) for 2 days. Tissue protein lysate was probed with an anti-pan-ERK antibody (EMD Millipore, Cat# ABS44, 1:50 dilution). The relative abundance of each phospho-isoform of ERK1 and ERK2 was quantified as fraction of total ERK1 or ERK2, respectively. Error bars: Standard error of the mean across three technical replicates. FIG. 9F Expression level of cleaved PARP-1 (apoptosis marker) quantified by NIA size assay in tissue slice cultures (TSCs) from human kidney cancer tissue expanded in mice as patient-derived xenograft. Slices of tumor tissue from PDX mice were cultured in vitro and treated with either IQGAP1 WW domain peptide (WW), scrambled peptide (scr.; negative control for WW), trametinib (MEK inhibitor) or vehicle (DMSO; negative control for trametinib) for 2 days. Tissue protein lysate was probed with an anti-cl. PARP-1 antibody (EMD Millipore, Cat# ABC26, 1:100 dilution) and the expression level quantified by normalization to tubulin (‘housekeeping’ gene). K562: cell line control, untreated (negative control) or treated with imatinib (+ imat.; positive control) at 10 μM for 29 h. FIG. 9G Expression level of PCNA and Cyclin D1 (proliferation markers) quantified by NIA size assay in tissue slice cultures (TSCs) from human kidney cancer tissue expanded in mice as patient-derived xenograft. Slices of tumor tissue from PDX mice were cultured in vitro and treated with either IQGAP1 WW domain peptide (WW), scrambled peptide (scr.; negative control for WW), trametinib (MEK inhibitor) or vehicle (DMSO; negative control for trametinib) for 2 days. Tissue protein lysate was probed with anti-PCNA (Cell Signaling Technology, clone PC10, Cat#2586, 1:50 dilution) or anti-Cyclin D1 antibody (Thermo Scientific, clone SP4, Cat# RM-9104-S0) and the expression level quantified by normalization to tubulin (‘housekeeping’ gene). K562: cell line control, untreated (positive control) or treated with imatinib (+ imat.; negative control) at 10 μM for 29 h. NIA could be used to measure a panel of apoptosis and proliferation markers in human cancer tissue, treated ex vivo, to compare how drugs work or don't work: trametinib decreased cell cycle proliferation (PCNA) and did not change apoptosis (cleaved PARP-1), whereas WW peptide induced both apoptosis and increased cell cycle (PCNA).

FIG. 10A-10B. FIG. 10A MYC inactivation increases pErk1, ppErk2, and Erk2 levels in T-ALL 4188 cell line. Twenty four hour MYC inactivation by treatment of tetracycline in T-ALL mouse-derived cell line 4188 (green) compared to high MYC (blue) shows phospho-Erk changes as detected by NIA charge assay. FIG. 10B MYC inactivation decreases Erk1 level in Burkitt P493-6 cell line. Twenty four hour MYC inactivation by treatment of tetracycline in human Burkitt lymphoma cell line P493-6 (green) compared to high MYC (blue) and Hela cells (grey) shows Erk1 decrease upon MYC inactivation as detected by Nano Immuno size assay.

FIG. 11: NIA can be used to measure drug targets PDL1 and VEGFR2. Left: SU-DHL-1 cell line (positive control, ‘+’) and RCC-4 cell line (negative control, ‘-’) were probed for PD-L1 with anti-human PDL1 antibody (Abcam, clone 28-8, Cat# ab205921, 1:50 dilution). PD-L1 is specifically detected in the positive control cell line. Right: TIME cell line (strong expression of VEGFR2, ‘+’) and RCC-4 cell line (weak expression of VEGFR2, ‘−’) were probed for VEGFR2 with anti-human VEGFR2 antibody (Cell Signaling Technology, clone 55611, Cat#2479, 1:50 dilution). Higher levels of VEGFR2 are detected in TIME cells compared to RCC-4.

### DEFINITIONS

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “and”, and “the” include plural referents unless the context clearly dictates otherwise. Thus, for example, reference to “a compound” includes a plurality of such compounds and reference to “the agent” includes reference to one or more agents and equivalents thereof known to those skilled in the art, and so forth. All technical and scientific terms used herein have the same meaning as commonly understood to one of ordinary skill in the art to which this invention belongs unless clearly indicated otherwise.

NIA.

In some embodiments methods are provided for nanoimmunoassay (NIA), including the serial analysis of cancer. NIA detection accurately measure oncoprotein expression and activation in limited clinical specimens, including particularly protein isoforms that differ in phosphorylation. The NIA detection method combines isoelectric protein focusing and antibody detection in a microfluidic system. Proteins, including isoforms of proteins, may be detected by NIA isoelectric focusing or size separation.

Samples may be taken at a single timepoint, or may be taken at multiple timepoints, for example, taken from at least two timepoints. Samples may be as small as 100,000 cells, as small as 5000 cells, as small as 1000 cells, as small as 500 cells, as small as 100 cells, as small as 50 cells or less. In some embodiments, the sample is less than 1000 cells, such as less than 500 cells, 400 cells, 300 cells, 200 cells, 100 cells, 90 cells, 80 cells, 70 cells, 60 cells, 50 cells, 40 cells, 30 cells, 20 cells, or 10 cells. In some embodiments the sample is a fine needle aspirate, (FNA). FNAs are performed at physicians' discretion. This procedure entails inserting a small-gauge needle, usually a 21- to 25-gauge needle, into a mass to remove a cellular sample for microscopic evaluation. The procedure should be performed by using one or more sewing machine-like excursions, while applying minimal negative pressure. In some embodiments, no more than 0.5 cc of suction is needed.

The term “sample” with respect to a patient encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as cancer cells. The definition also includes sample that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's cancer cell, e.g., a sample comprising polynucleotides and/or polypeptides that is obtained from a patient's cancer cell (e.g., a cell lysate or other cell extract comprising polynucleotides and/or polypeptides); and a sample comprising cancer cells from a patient. A biological sample comprising a cancer cell from a patient can also include non-cancerous cells.

Biopsy samples. Biopsy samples, and particularly fine needle aspirates (FNA) can be maintained at approximately 4°, and up to room temperature, e.g. at around about 25° C. following obtention from a patient for a period of up to 3 days, up to 2 days, up to 1 day, up to 12 hours, up to 6 hours. The sample may be maintained without lysis of cells or red blood cells. The sample, or a lysate thereof, is stable when stored frozen at about −80 degrees C. for an extended period of time. Thus in some embodiments of the invention the analysis is performed on a previously frozen sample.

The cells, which may be cells after exposure to an agent or condition of interest, are lysed prior to analysis. Methods of lysis are known in the art, including sonication, non-ionic surfactants, etc. Non-ionic surfactants include the Triton™ family of detergents, e.g. Triton™ X-15; Triton™ X-35; Triton™ X-45; Triton™ X-100; Triton™ X-102; Triton™ X-114; Triton™ X-165, etc. Brij™ detergents are also similar in structure to Triton™ X detergents in that they have varying lengths of polyoxyethylene chains attached to a hydrophobic chain. The Tween™ detergents are nondenaturing, nonionic detergents, which are polyoxyethylene sorbitan esters of fatty acids. Tween™ 80 is derived from oleic acid with a C₁₈ chain while Tween™ 20 is derived from lauric acid with a C₁₂ chain. The zwitterionic detergent, CHAPS, is a sulfobetaine derivative of cholic acid. BICINE (diethylolglycine) is zwitterionic amino acid buffer that may be formulated with CHAPS. This zwitterionic detergent and buffer is useful for membrane protein solubilization when protein activity is important. The surfactant is contacted with the cells for a period of time sufficient to lyse the cells and remove additional adherent cells from the system.

Methods of cellular fractionation are also known in the art. Subcellular fractionation consists of two major steps, disruption of the cellular organization (lysis) and fractionation of the homogenate to separate the different populations of organelles. Such a homogenate can then be resolved by differential centrifugation into several fractions containing mainly (1) nuclei, heavy mitochondria, cytoskeletal networks, and plasma membrane; (2) light mitochondria, lysosomes, and peroxisomes; (3) Golgi apparatus, endosomes and microsomes, and endoplasmic reticulum (ER); and (4) cytosol. Each population of organelles is characterized by size, density, charge, and other properties on which the separation relies.

The isoelectric focused or size separated protein is bound to a specific binding member. For relative ratio measurements, a single, pan-specific antibody that recognizes all isoforms of the protein may be used, for example pan-specific antibody, etc. The total amount of the protein, e.g. ERK2, Erk1, etc. is determined, and NIA is used to calculate the percent that is phosphorylated. NIA generates peaks, and the area of each peak was calculated by dropping verticals to the baseline at the peak start and end, and summing the area between the start and endpoints. For normalized value measurements a similar process is used, but in addition the assay utilizes an antibody for the protein of interest, e.g. pan-specific antibody, and a loading control antibody, e.g. HSP-70 antibody, tubulin and the like, for normalization. NIA is utilized to discriminate the different isoforms.

Comparisons may be performed between tissue suspected of being a tumor tissue and a paired normal or on-tumor control tissue, e.g. a suspected tumor sample, v. an adjacent non-tumor skin sample, and the like. Comparisons may also be performed with reference tumor tissue, with a time series of samples, e.g. before and after treatment, and the like. A ratio may be non-tumor/tumor, or tumor/non-tumor. In some embodiments a ratio provides a more predictive or diagnostic biomarker than a single measurement of tumor or normal.

In some embodiments NIA is used to monitor changes in phosphorylation. The vast majority of phosphorylations occur as a mechanism to regulate the biological activity of a protein and as such are transient. In animal cells serine, threonine and tyrosine are the amino acids subject to phosphorylation. The largest group of kinases are those that phosphorylate either serines or threonines and as such are termed serine/threonine kinases. The ratio of phosphorylation of the three different amino acids is approximately 1000/100/1 for serine/threonine/tyrosine. Although the level of tyrosine phosphorylation is minor, the importance of phosphorylation of this amino acid is profound. As an example, the activity of numerous growth factor receptors is controlled by tyrosine phosphorylation.

A “patient” for the purposes of the present invention includes both humans and other animals, particularly mammals, including pet and laboratory animals, e.g. mice, rats, rabbits, etc. Thus the methods are applicable to both human therapy and veterinary applications. In one embodiment the patient is a mammal, preferably a primate. In other embodiments the patient is human.

The terms “subject,” “individual,” and “patient” are used interchangeably herein to refer to a mammal being assessed for treatment and/or being treated. In an embodiment, the mammal is a human. The terms “subject,” “individual,” and “patient” encompass, without limitation, individuals having cancer. Subjects may be human, but also include other mammals, particularly those mammals useful as laboratory models for human disease, e.g. mouse, rat, etc.

The terms “cancer,” “neoplasm,” and “tumor” are used interchangeably herein to refer to cells which exhibit autonomous, unregulated growth, such that they exhibit an aberrant growth phenotype characterized by a significant loss of control over cell proliferation. Cells of interest for detection, analysis, or treatment in the present application include precancerous (e.g., benign), malignant, pre-metastatic, metastatic, and non-metastatic cells. Cancers of virtually every tissue are known. The phrase “cancer burden” refers to the quantum of cancer cells or cancer volume in a subject. Reducing cancer burden accordingly refers to reducing the number of cancer cells or the cancer volume in a subject. The term “cancer cell” as used herein refers to any cell that is a cancer cell or is derived from a cancer cell e.g. clone of a cancer cell. Many types of cancers are known to those of skill in the art, including solid tumors such as carcinomas, sarcomas, glioblastomas, melanomas, lymphomas, myelomas, etc., and circulating cancers such as leukemias. Examples of cancer include but are not limited to, ovarian cancer, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, and brain cancer.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

As used herein, the terms “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs; therefore tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part which is not directly connected to the organ of the original cancerous tumor. Metastasis can also be defined as several steps of a process, such as the departure of cancer cells from an original tumor site, and migration and/or invasion of cancer cells to other parts of the body.

The term “diagnosis” is used herein to refer to the identification of a molecular or pathological state, disease or condition, such as the identification of a molecular subtype of breast cancer, prostate cancer, or other type of cancer.

The term “prognosis” is used herein to refer to the prediction of the likelihood of cancer-attributable death or progression, including recurrence, metastatic spread, and drug resistance, of a neoplastic disease, such as ovarian cancer. The term “prediction” is used herein to refer to the act of foretelling or estimating, based on observation, experience, or scientific reasoning. In one example, a physician may predict the likelihood that a patient will survive, following surgical removal of a primary tumor and/or chemotherapy for a certain period of time without cancer recurrence.

As used herein, the terms “treatment,” “treating,” and the like, refer to administering an agent, or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of a tumor in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease.

Treating may refer to any indicia of success in the treatment or amelioration or prevention of an cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with cancer or other diseases. The term “therapeutic effect” refers to the reduction, elimination, or prevention of the disease, symptoms of the disease, or side effects of the disease in the subject.

Examples of therapeutic agents identified in the art as useful in the treatment of neoplastic disease, include without limitation, abitrexate, adriamycin, adrucil, amsacrine, asparaginase, anthracyclines, azacitidine, azathioprine, bicnu, blenoxane, busulfan, bleomycin, camptosar, camptothecins, carboplatin, carmustine, cerubidine, chlorambucil, cisplatin, cladribine, cosmegen, cytarabine, cytosar, cyclophosphamide, cytoxan, dactinomycin, docetaxel, doxorubicin, daunorubicin, ellence, elspar, epirubicin, etoposide, fludarabine, fluorouracil, fludara, gemcitabine, gemzar, hycamtin, hydroxyurea, hydrea, idamycin, idarubicin, ifosfamide, ifex, irinotecan, lanvis, leukeran, leustatin, matulane, mechlorethamine, mercaptopurine, methotrexate, mitomycin, mitoxantrone, mithramycin, mutamycin, myleran, mylosar, navelbine, nipent, novantrone, oncovin, oxaliplatin, paclitaxel, paraplatin, pentostatin, platinol, plicamycin, procarbazine, purinethol, ralitrexed, taxotere, taxol, teniposide, thioguanine, tomudex, topotecan, valrubicin, velban, vepesid, vinblastine, vindesine, vincristine, vinorelbine, VP-16, and vumon.

Targeted therapeutics may include tyrosine-kinase inhibitors, such as Imatinib mesylate (Gleevec, also known as STI-571), Gefitinib (Iressa, also known as ZD1839), Erlotinib (marketed as Tarceva), Sorafenib (Nexavar), Sunitinib (Sutent), Dasatinib (Sprycel), Lapatinib (Tykerb), Nilotinib (Tasigna), and Bortezomib (Velcade), Jakafi (ruxolitinib); Janus kinase inhibitors, such as tofacitinib; ALK inhibitors, such as crizotinib; Bcl-2 inhibitors, such as obatoclax, venclexta, and gossypol; FLT3 inhibitors, such as midostaurin (Rydapt), IDH inhibitors, such as AG-221, PARP inhibitors, such as Iniparib and Olaparib; P13K inhibitors, such as perifosine; VEGF Receptor 2 inhibitors, such as Apatinib; AN-152 (AEZS-108) doxorubicin linked to [D-Lys(6)]-LHRH; Braf inhibitors, such as vemurafenib, dabrafenib, and LGX818; MEK inhibitors, such as trametinib; CDK inhibitors, such as PD-0332991 and LEE011; Hsp90 inhibitors, such as salinomycin; and/or small molecule drug conjugates, such as Vintafolide; serine/threonine kinase inhibitors, such as Temsirolimus (Torisel), Everolimus (Afinitor), Vemurafenib (Zelboraf), Trametinib (Mekinist), and Dabrafenib (Tafinlar).

Examples of biological agents identified in the art as useful in the treatment of neoplastic disease, include without limitation, cytokines or cytokine antagonists such as IL-12, INFα, or anti-epidermal growth factor receptor, radiotherapy, irinotecan; tetrahydrofolate antimetabolites such as pemetrexed; antibodies against tumor antigens, a complex of a monoclonal antibody and toxin, a T-cell adjuvant, bone marrow transplant, or antigen presenting cells (e.g., dendritic cell therapy), anti-tumor vaccines, replication competent viruses, signal transduction inhibitors (e.g., Gleevec® or Herceptin®) or an immunomodulator to achieve additive or synergistic suppression of tumor growth, cyclooxygenase-2 (COX-2) inhibitors, steroids, TNF antagonists (e.g., Remicade® and Enbrel®), interferon-β1a (Avonex®), and interferon-β1b (Betaseron®) as well as combinations of one or more of the foregoing as practiced in known chemotherapeutic treatment regimens readily appreciated by the skilled clinician in the art.

Tumor specific monoclonal antibodies may include, without limitation, Rituximab (marketed as MabThera or Rituxan), Alemtuzumab, Panitumumab, Ipilimumab (Yervoy), etc.

In some embodiments the treatment includes immune checkpoint therapy. Examples of immune checkpoint therapies include inhibitors of the binding of PD1 to PDL1 and/or PDL2. PD1 to PDL1 and/or PDL2 inhibitors are well known in the art. Examples of commercially available monoclonal antibodies that interfere with the binding of PD1 to PDL1 and/or PDL2 include nivolumab (Opdivo®, BMS-936558, MDX1106, commercially available from BristolMyers Squibb, Princeton N.J.), pembrolizumab (Keytruda®MK-3475, lambrolizumab, commercially available from Merck and Company, Kenilworth N.J.), and atezolizumab (Tecentriq®, Genentech/Roche, South San Francisco Calif.). Additional examples of PD1 inhibitory antibodies include but are not limited to durvalumab (MED14736, Medimmune/AstraZeneca), pidilizumab (CT-011, CureTech), PDR001 (Novartis), BMS-936559 (MDX1105, Bristol Myers Squibb), and avelumab (MSB0010718C, Merck Serono/Pfizer) and SHR-1210 (Incyte). Additional antibody PD1 pathway inhibitors are described in U.S. Pat. No. 8,217,149 (Genentech, Inc) issued Jul. 10, 2012; U.S. Pat. No. 8,168,757 (Merck Sharp and Dohme Corp.) issued May 1, 2012, U.S. Pat. No. 8,008,449 (Medarex) issued Aug. 30, 2011, U.S. Pat. No. 7,943,743 (Medarex, Inc) issued May 17, 2011. Additionally, small molecule PD1 to PDL1 and/or PDL2 inhibitors are known in the art. See, e.g. Sasikumar, et al as WO2016142833A1 and Sasikumar, et al. WO2016142886A2, BMS-1166 and BMS-1001 (Skalniak, et al (2017) Oncotarget 8(42): 72167-72181).

Of particular interest are targeted therapeutics, which may include Cabozantinib, sold under the brand-name Cabometyx and Cometriq, which is a medication used to treat medullary thyroid cancer and a second line treatment for renal cell carcinoma among others. It is a small molecule inhibitor of the tyrosine kinases c-Met and VEGFR2, and also inhibits AXL and RET. Axitinib is a small molecule tyrosine kinase inhibitor developed by Pfizer. It has been shown to significantly inhibit growth of breast cancer in animal (xenograft) models and has shown partial responses in clinical trials with renal cell carcinoma (RCC) and several other tumour types. Trametinib is a MEK inhibitor drug with anti-cancer activity. It inhibits MEK1 and MEK2. Trametinib had good results for metastatic melanoma carrying the BRAF V600E mutation. Rigosertib is a small molecule agent, which is believed to block cellular signaling by targeting RAS effector pathways.

“In combination with”, “combination therapy” and “combination products” refer, in certain embodiments, to the concurrent administration to a patient of a first therapeutic and the compounds as used herein. When administered in combination, each component can be administered at the same time or sequentially in any order at different points in time. Thus, each component can be administered separately but sufficiently closely in time so as to provide the desired therapeutic effect.

“Concomitant administration” of a cancer therapeutic drug, immune-oncology agent, tumor-directed antibody, etc. in combination with another agent means administration at such time that both the drug, antibody and the composition of the present invention will have a therapeutic effect. Such concomitant administration may involve concurrent (i.e. at the same time), prior, or subsequent administration of the drug, or antibody with respect to the administration of a compound of the invention. A person of ordinary skill in the art would have no difficulty determining the appropriate timing, sequence and dosages of administration for particular drugs and compositions of the present invention.

As used herein, endpoints for treatment will be given a meaning as known in the art and as used by the Food and Drug Administration.

Overall survival is defined as the time from randomization until death from any cause, and is measured in the intent-to-treat population. Survival is considered the most reliable cancer endpoint, and when studies can be conducted to adequately assess survival, it is usually the preferred endpoint. This endpoint is precise and easy to measure, documented by the date of death. Bias is not a factor in endpoint measurement. Survival improvement should be analyzed as a risk-benefit analysis to assess clinical benefit. Overall survival can be evaluated in randomized controlled studies. Demonstration of a statistically significant improvement in overall survival can be considered to be clinically significant if the toxicity profile is acceptable, and has often supported new drug approval. A benefit of the methods of the invention can include increased overall survival of patients.

Endpoints that are based on tumor assessments include DFS, ORR, TTP, PFS, and time-to-treatment failure (TTF). The collection and analysis of data on these time-dependent endpoints are based on indirect assessments, calculations, and estimates (e.g., tumor measurements). Disease-Free Survival (DFS) is defined as the time from randomization until recurrence of tumor or death from any cause. The most frequent use of this endpoint is in the adjuvant setting after definitive surgery or radiotherapy. DFS also can be an important endpoint when a large percentage of patients achieve complete responses with chemotherapy.

Objective Response Rate. ORR is defined as the proportion of patients with tumor size reduction of a predefined amount and for a minimum time period. Response duration usually is measured from the time of initial response until documented tumor progression. Generally, the FDA has defined ORR as the sum of partial responses plus complete responses. When defined in this manner, ORR is a direct measure of drug antitumor activity, which can be evaluated in a single-arm study.

Time to Progression and Progression-Free Survival. TTP and PFS have served as primary endpoints for drug approval. TTP is defined as the time from randomization until objective tumor progression; TTP does not include deaths. PFS is defined as the time from randomization until objective tumor progression or death. The precise definition of tumor progression is important and should be carefully detailed in the protocol.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

“Pharmaceutically acceptable salts and esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g., ethanolamine, diethanolamine, triethanolamine, tromethamine, N methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g., C₁₋₆ alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention may be present in more than one stereoisomeric form, and the naming of such compounds is intended to include all single stereoisomers and all mixtures (whether racemic or otherwise) of such stereoisomers.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” means the amount that, when administered to a subject for treating a disease, is sufficient to effect treatment for that disease.

### Methods

Methods are provided for analysis, diagnosis and treating or reducing growth of primary or metastatic cancer. Cancer cells can be distinguished from normal counterpart tissue through detecting specific patterns of proteins present in the cells, including patterns of different isoforms, e.g. phosphorylated forms of a protein. Such proteins may be a component of signaling pathways that regulate cell growth, replication, metastasis, and the like.

Individuals may be monitored for treatment and/or selected for therapy by determining the phenotype of the cancer cells. In one embodiment a nanofluidic proteomic immunoassay (NIA) is applied to quantify phosphoisoforms in a small amount of lysate from a tumor.

In some embodiments a specific change in isoforms is correlated with response to a therapeutic agent. Assessment responsiveness allows improved care by indicating where therapeutic action may be required, at an early stage of disease. Where an individual is analyzed and found to be responsive to a therapy, treatment may be continued, or the patient allowed to recover following a suitable response. Where an individual is treated and found to not be responsive, an alternative therapy is administered.

In some embodiments an individual is assessed for responsiveness to a therapeutic agent by administering a therapeutic dose of an agent, e.g. a cancer chemotherapeutic agent. Following a period of time sufficient for response, e.g. at 12 hours, at least 18 hours, at least 24 hours, up to 3 weeks or more, up to 2 weeks, up to 10 days, up to 7 days, up to 5 days, up to 3 days, up to 48 hours, up to 24 hours, a biopsy sample is obtained. The biopsy sample is optionally frozen and stored at −80C prior to analysis.

The sample is then analyzed by NIA for the protein isoforms of interest. The pattern of isoform distribution in the treated tumor cell may be compared to normal cells, reference cancer cells, and the like. In some embodiments adjacent tissue samples are obtained, e.g. two tumor samples, a tumor sample and counterpart normal tissue, etc. In some embodiments a time series of tumor tissues are obtained, e.g. 2, 3, 4 or more samples from a single tumor. In some embodiments a sample is compared to a reference sample, e.g. an isoform pattern of a cell known to be responding to a therapy.

Multiple samples may be obtained and analyzed from an individual over time, including an individual treated with a therapeutic regimen for treatment of the cancer. Multiple samples may also be obtained and analyzed over a patient cohort group, for example in the context of clinical trials.

In some embodiments, the isoform distribution is determined for a pre- and post-treatment matched sample, usually with matched controls. Samples of known disease cases may be divided into post-treatment time intervals and logistic regression analysis performed to determine which of the time intervals the isoform distribution is most strongly associated with.

The protein distribution pattern may be generated from a biological sample using any convenient NIA protocol. The readout may be a mean, average, median or the variance or other statistically or mathematically-derived value associated with the measurement. The readout information may be further refined by direct comparison with the corresponding reference or control pattern. A pattern may be evaluated on a number of points: to determine if there is a statistically significant change at any point in the data matrix; whether the change is an increase or decrease in prevalence of an isoform; and the like. The absolute values will display a variability that is inherent in live biological systems.

Following obtainment of the protein distribution pattern from the sample being assayed, it is compared with a reference or control profile to make an analysis regarding the phenotype of the patient from which the sample was obtained/derived, for example whether the individual is responsive to treatment. Typically a comparison is made with a sample or set of samples as described above. Additionally, a reference or control signature pattern may be a signature pattern that is obtained from a sample known to be responsive, and therefore may be a positive reference or control profile.

In certain embodiments, the obtained protein distribution pattern is compared to a single reference/control profile to obtain information regarding the phenotype of the patient being assayed. In yet other embodiments, the obtained protein distribution pattern is compared to two or more different reference/control profiles to obtain more in depth information regarding the phenotype of the patient. For example, the obtained protein distribution pattern may be compared to a positive and negative reference profile to obtain confirmed information regarding whether the patient has the phenotype of interest.

An algorithm that will discriminate robustly between individuals in different classifications with respect to responsiveness to therapy, and controls for confounding variables and evaluating potential interactions is used for prognostic purposes.

The protein distribution pattern is determined by the methods described above. The quantitative data thus obtained is then subjected to an analytic classification process. In such a process, the raw data is manipulated according to an algorithm, where the algorithm has been pre-defined by a training set of data, for example as described in the examples provided herein.

An algorithm may utilize the training set of data provided herein, or may utilize the guidelines provided herein to generate an algorithm with a different set of data.

An analytic classification process may use any one of a variety of statistical analytic methods to manipulate the quantitative data and provide for classification of the sample. Examples of useful methods include linear discriminant analysis, recursive feature elimination, a prediction analysis of microarray, a logistic regression, a CART algorithm, a FlexTree algorithm, a LART algorithm, a random forest algorithm, a MART algorithm, machine learning algorithms; etc. Using any one of these methods, a protein distribution pattern may be used to generate a predictive model. In the generation of such a model, a dataset comprising control, diseased, responsive to treatment, non-responsive to treatment, etc. samples may be used as a training set. A training set will contain data for one or more different isoform distributions of interest.

Classification can be made according to predictive modeling methods that set a threshold for determining the probability that a sample belongs to a given class, i.e. responsive, non-responsive, etc. The probability preferably is at least 50%, or at least 60% or at least 70% or at least 80% or higher. Classifications also may be made by determining whether a comparison between an obtained protein distribution pattern and a reference protein distribution pattern yields a statistically significant difference. If such a comparison is not statistically significantly different from the reference protein distribution pattern, then the sample from which the protein distribution pattern was obtained is classified as belonging to the reference protein distribution pattern class.

The predictive ability of a model may be evaluated according to its ability to provide a quality metric, e.g. AUC or accuracy, of a particular value, or range of values. In some embodiments, a desired quality threshold is a predictive model that will classify a sample with an accuracy of at least about 0.7, at least about 0.75, at least about 0.8, at least about 0.85, at least about 0.9, at least about 0.95, or higher. As an alternative measure, a desired quality threshold may refer to a predictive model that will classify a sample with an AUC (area under the curve) of at least about 0.7, at least about 0.75, at least about 0.8, at least about 0.85, at least about 0.9, or higher.

As is known in the art, the relative sensitivity and specificity of a predictive model can be “tuned” to favor either the selectivity metric or the sensitivity metric, where the two metrics have an inverse relationship. The limits in a model as described above can be adjusted to provide a selected sensitivity or specificity level, depending on the particular requirements of the test being performed. One or both of sensitivity and specificity may be at least about at least about 0.7, at least about 0.75, at least about 0.8, at least about 0.85, at least about 0.9, or higher.

The raw data may be initially analyzed by measuring the values for each marker, usually in triplicate or in multiple triplicates. The data may be manipulated, for example, raw data may be transformed using standard curves, and the average of triplicate measurements used to calculate the average and standard deviation for each patient. These values may be transformed before being used in the models, e.g. log-transformed, Box-Cox transformed (see Box and Cox (1964) J. Royal Stat. Soc., Series B, 26:211-246), etc. The data are then input into a predictive model, which will classify the sample according to the state. The resulting information may be transmitted to a patient or health professional.

In one embodiment, hierarchical clustering is performed in the derivation of a predictive model, where the Pearson correlation is employed as the clustering metric. One approach is to consider a patient protein distribution pattern as a “learning sample” in a problem of “supervised learning”. CART is a standard in applications to medicine (Singer (1999) Recursive Partitioning in the Health Sciences, Springer), which may be modified by transforming any qualitative features to quantitative features; sorting them by attained significance levels, evaluated by sample reuse methods for Hotelling's T² statistic; and suitable application of the lasso method. Problems in prediction are turned into problems in regression without losing sight of prediction, indeed by making suitable use of the Gini criterion for classification in evaluating the quality of regressions.

This approach has led to what is termed FlexTree (Huang (2004) PNAS 101:10529-10534). FlexTree has performed very well in simulations and when applied to SNP and other forms of data. Software automating FlexTree has been developed. Alternatively LARTree or LART may be used, see Turnbull (2005) Classification Trees with Subset Analysis Selection by the Lasso, Stanford University. The name reflects binary trees, as in CART and FlexTree; the lasso, as has been noted; and the implementation of the lasso through what is termed LARS by Efron et al. (2004) Annals of Statistics 32:407-451. See, also, Huang et al. (2004) Tree-structured supervised learning and the genetics of hypertension. Proc Natl Acad Sci USA. 101(29):10529-34.

Other methods of analysis that may be used include logic regression. One method of logic regression Ruczinski (2003) Journal of Computational and Graphical Statistics 12:475-512. Logic regression resembles CART in that its classifier can be displayed as a binary tree. It is different in that each node has Boolean statements about features that are more general than the simple “and” statements produced by CART.

Another approach is that of nearest shrunken centroids (Tibshirani (2002) PNAS 99:6567-72). The technology is k-means-like, but has the advantage that by shrinking cluster centers, one automatically selects features (as in the lasso) so as to focus attention on small numbers of those that are informative. The approach is available as PAM software and is widely used. Two further sets of algorithms are random forests (Breiman (2001) Machine Learning 45:5-32 and MART (Hastie (2001) The Elements of Statistical Learning, Springer). These two methods are already “committee methods.” Thus, they involve predictors that “vote” on outcome.

To provide significance ordering, the false discovery rate (FDR) may be determined. First, a set of null distributions of dissimilarity values is generated. In one embodiment, the values of observed protein distribution pattern are permuted to create a sequence of distributions of correlation coefficients obtained out of chance, thereby creating an appropriate set of null distributions of correlation coefficients (see Tusher et al. (2001) PNAS 98, 5116-21, herein incorporated by reference). The set of null distribution is obtained by: permuting the values of each protein distribution pattern for all available protein distribution pattern; calculating the pair-wise correlation coefficients for all protein distribution pattern; calculating the probability density function of the correlation coefficients for this permutation; and repeating the procedure for N times, where N is a large number, usually 300. Using the N distributions, one calculates an appropriate measure (mean, median, etc.) of the count of correlation coefficient values that their values exceed the value (of similarity) that is obtained from the distribution of experimentally observed similarity values at given significance level.

The FDR is the ratio of the number of the expected falsely significant correlations (estimated from the correlations greater than this selected Pearson correlation in the set of randomized data) to the number of correlations greater than this selected Pearson correlation in the empirical data (significant correlations). This cut-off correlation value may be applied to the correlations between experimental protein distribution pattern.

Using the aforementioned distribution, a level of confidence is chosen for significance. This is used to determine the lowest value of the correlation coefficient that exceeds the result that would have obtained by chance. Using this method, one obtains thresholds for positive correlation, negative correlation or both. Using this threshold(s), the user can filter the observed values of the pairwise correlation coefficients and eliminate those that do not exceed the threshold(s). Furthermore, an estimate of the false positive rate can be obtained for a given threshold. For each of the individual “random correlation” distributions, one can find how many observations fall outside the threshold range. This procedure provides a sequence of counts. The mean and the standard deviation of the sequence provide the average number of potential false positives and its standard deviation.

The selection of a number of informative isoforms for building classification models may utilize the definition of a performance metric and a user-defined threshold for producing a model with useful predictive ability for individual responsiveness to therapy based on this metric. For example, the performance metric may be the AUC, the sensitivity and/or specificity of the prediction as well as the overall accuracy of the prediction model.

Also provided are databases and computer systems for analysis. Databases can typically comprise distribution pattern information from various conditions, such as responses of cells to a variety of treatments. The results and databases thereof may be provided in a variety of media to facilitate their use.

“Media” can refer to a manufacture that contains the distribution pattern information; and methods of analysis as described above. The databases and comparative algorithms can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc.

As used herein, “a computer-based system” refers to the hardware means, software means, and data storage means used to analyze the information provided herein. The minimum hardware of the computer-based systems of the present invention comprises a central processing unit (CPU), input means, output means, and data storage means. A skilled artisan can readily appreciate that any one of the currently available computer-based system are suitable for use in analysis. The data storage means may comprise any manufacture comprising a recording of the present information as described above, or a memory access means that can access such a manufacture.

A variety of structural formats for the input and output means can be used to input and output the information in the computer-based systems of the present invention. Such presentation provides a skilled artisan with a ranking of similarities and identifies the degree of similarity contained in the test expression repertoire.

The data analysis may be implemented in hardware or software, or a combination of both. In one embodiment, a machine-readable storage medium is provided, the medium comprising a data storage material encoded with machine readable data which, when using a machine programmed with instructions for using said data, is capable of displaying a any of the datasets and data comparisons of this invention. Such data may be used for a variety of purposes, such as drug discovery, analysis of interactions between cellular components, and the like. In some embodiments, the analysis is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language may be a compiled or interpreted language. Each such computer program can be stored on a storage media or device (e.g., ROM or magnetic diskette) readable by a general or special purpose programmable computer, for configuring and operating the computer when the storage media or device is read by the computer to perform the procedures described herein. The system may also be considered to be implemented as a computer-readable storage medium, configured with a computer program, where the storage medium so configured causes a computer to operate in a specific and predefined manner to perform the functions described herein.

A variety of structural formats for the input and output means can be used to input and output the information in the computer-based systems. One format for an output tests datasets possessing varying degrees of similarity to a trusted repertoire. Such presentation provides a skilled artisan with a ranking of similarities and identifies the degree of similarity contained in the test repertoire.

Further provided herein is a method of storing and/or transmitting, via computer, data collected by the methods disclosed herein. Any computer or computer accessory including, but not limited to software and storage devices, can be utilized to practice the present invention. Data can be input into a computer by a user either directly or indirectly. Additionally, any of the devices which can be used to perform or analyze NIA can be linked to a computer, such that the data is transferred to a computer and/or computer-compatible storage device. Data can be stored on a computer or suitable storage device (e.g., CD). Data can also be sent from a computer to another computer or data collection point via methods well known in the art (e.g., the internet, ground mail, air mail). Thus, data collected by the methods described herein can be collected at any point or geographical location and sent to any other geographical location.

Also provided are reagents and kits thereof for practicing one or more of the above-described methods. The subject reagents and kits thereof may vary greatly. Reagents of interest include reagents specifically designed for use in production of the above described protein distribution patterns associated with cancer cells and their responsiveness to therapy.

### EXPERIMENTAL

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### Example 1

### Impact of Time and Temperature of Specimen Storage on ERK Phosphorylation

Impact of specimen storage time on ERK phosphorylation. An important concern for the stability of ERK phosphorylation in clinical fine needle aspirate (FNA) specimens is the time interval that elapses between FNA harvesting and flash-freezing the FNA in the laboratory. We call this interval the FNA storage time. In addition, the temperature of FNA storage may affect ERK phosphorylation. To address these concerns, we performed benchmarking experiments to validate the stability of ERK phosphorylation in FNAs from patients with kidney cancer (see below). The relative abundances of phosphorylated and non-phosphorylated ERK isoforms are remarkably stable, suggesting that our measurements are not significantly altered by the method of FNA processing, even with a storage time up to 2 days in length.

For three FNAs from different patients, one portion of the FNA was processed and flash-frozen on the same day of tissue harvesting and the other portion stored at 4° C. in medium and processed either the next day or 2 days after harvesting. The relative abundances of ERK2 phospho-isoforms were measured by NIA in technical duplicate. As for the entire cohort, ERK1 showed little phosphorylation and was therefore excluded from this analysis.

Conclusion: The relative abundances of ERK2 phospho-isoforms show little change even after 2 days of storing FNAs in cold medium.

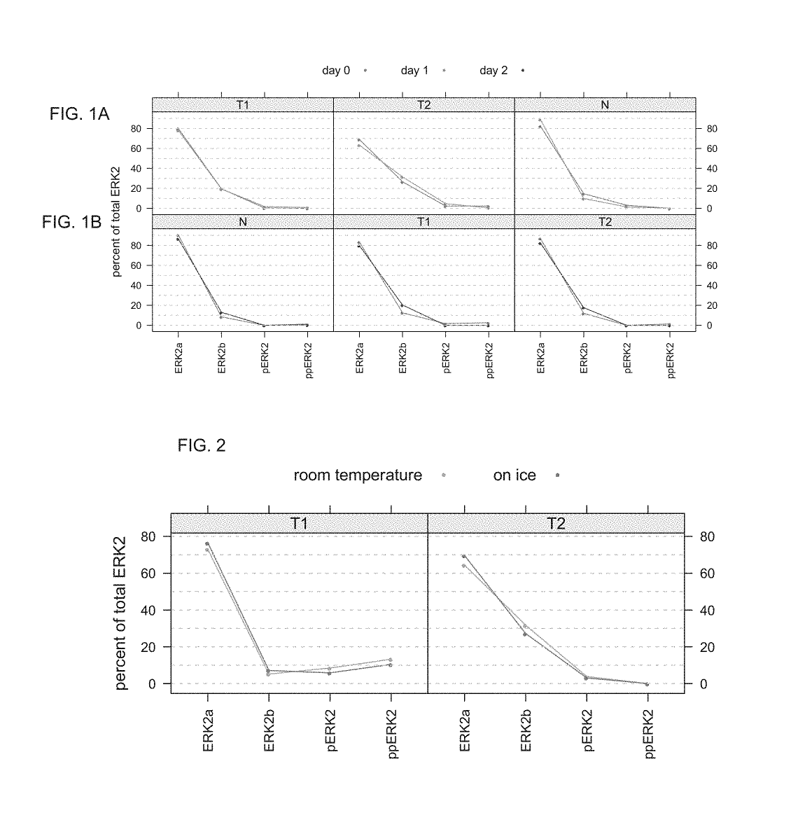

FIG. 1A and FIG. 1B show the impact of specimen storage temperature on ERK phosphorylation. For two FNAs from one tumor, transport and processing (resuspension, aliquoting, and centrifugation) was performed side-by-side at room temperature and on ice. The relative abundances of ERK2 phospho-isoforms were measured by NIA in technical duplicate and compared. As for the entire cohort, ERK1 showed little phosphorylation and was therefore excluded from this analysis.

Conclusion: The relative abundances of ERK2 phospho-isoforms show little difference between FNA transport and processing at room temperature vs. on ice up for at least 1 hour after harvesting.

### Methods

Tissue Acquisition and Clinical Parameters.

All patients of the urologic oncology clinic at the Stanford Cancer Institute undergoing radical or partial nephrectomy for renal masses were screened and cases for tissue collection were selected with representation of all clinical tumor stages (T1 through T4) and cytoreductive nephrectomies. Patients giving informed consent were enrolled on Stanford University IRB-approved research protocols allowing for the acquisition, banking, and molecular analysis of tissue. Kidney tumors from patients undergoing partial or radical nephrectomy were bivalved with the assistance of a pathologist immediately after surgical extirpation of the kidney. FNAs were performed using a 21-gauge needle on a 10 mL syringe, which was passed through the tissue 20 times under gentle negative pressure. FNAs were collected into RPMI-1640 medium (Gibco/Thermo Fisher Scientific, Waltham, Mass.), centrifuged, and the supernatant discarded. FNAs were transported either at room temperature or on ice, as described. Different transport times were tested. Pellets were flash frozen in liquid nitrogen and stored at −80° C. until batch analysis.

Nano-immuno assay (NIA). FNAs were lysed in Bicine/CHAPS buffer containing protease and phosphatase inhibitors (ProteinSimple, San Jose, Calif.) and analyzed in technical duplicate or triplicate using charge separation by isoelectric focusing in a microfluidic capillary on a NanoPro1000 or PeggySue Instrument (ProteinSimple). ERK isoforms were detected by chemiluminescence using a pan-ERK antibody (EMD Millipore, Cat. No. 06-182, Hayward, Calif.) and an HRP-conjugated anti-rabbit secondary antibody (ProteinSimple). Chemiluminescence intensity was measured over a range of exposure times (30 s to 960 s).

Quantification of chemiluminescence signal intensity. Quantification of the chemiluminescence signal intensity was done with Compass software (ProteinSimple). First, the exposure time for optimal signal-to-noise ratio within the dynamic range of the assay was determined, and background intensity was subtracted using a baseline fit. Then, the area under the fitted chemiluminescence intensity profile across the capillary was calculated for each fitted peak and its isoelectric point (pI) was determined with the help of internal pI standards (ProteinSimple). Lastly, peaks were assigned to specific ERK isoforms according to pI. Relative abundance of ERK2 phospho-isoforms was determined by determining ratios of areas of fitted peaks. FNA transport and processing is equivalent at room temperature vs. on ice.

### Example 2

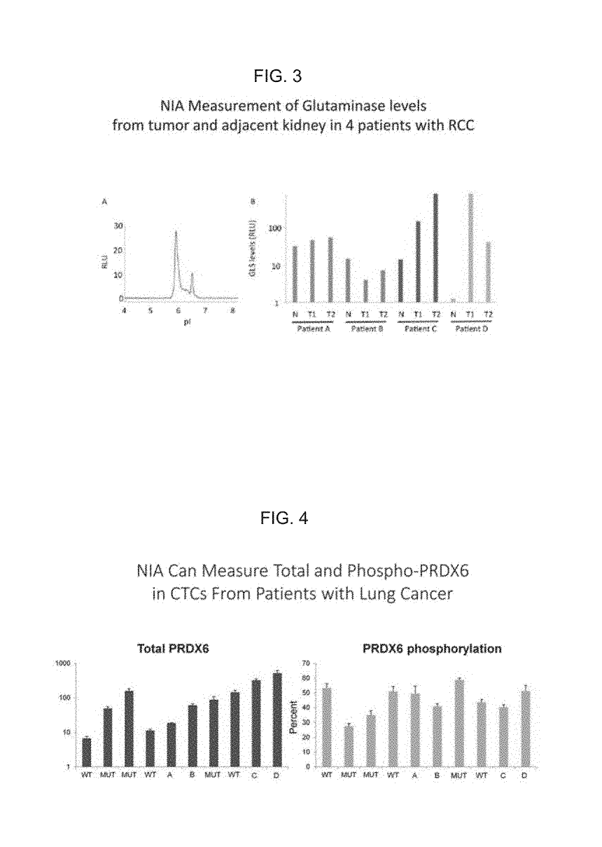

As shown in FIG. 3, fine needle aspirates of two regions of the same human RCC tumor (T1 and T2) and adjacent kidney tissue (N) were obtained from 4 patients with kidney cancer, and provide a distribution of glutaminase levels between samples. Charge-separation NIA was used to measure levels of both the KGA and GAC isoforms of glutaminase 1 (GLS1) in these samples using anti-GLS1 antibody. This demonstrated NIA analysis of protein isoforms involved in metabolism.

Shown in FIG. 4, NIA can measure the distribution of isoforms of the antioxidant protein Peroxiredoxin-6 (PRDX6) from frozen samples. Cells from each patient were analyzed with anti-PRDX6 antibody (Abcam, Cat#73350). Patients either had EGFR mutations (MUT), wild type (WT), or unknown EGFR status (A,B,C,D).

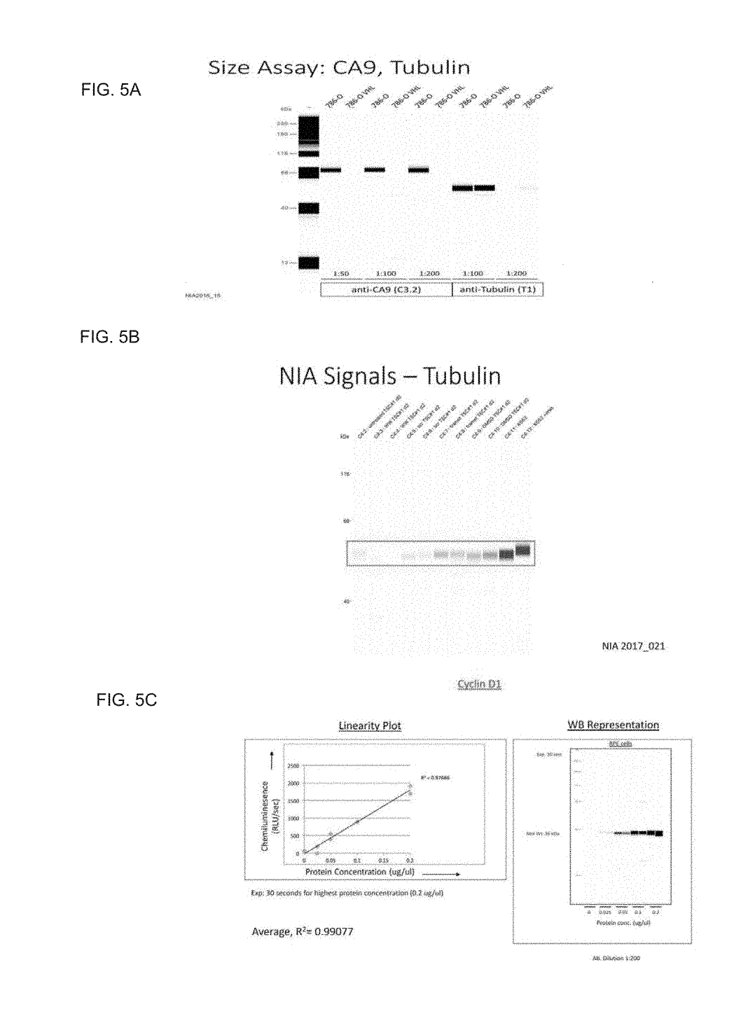

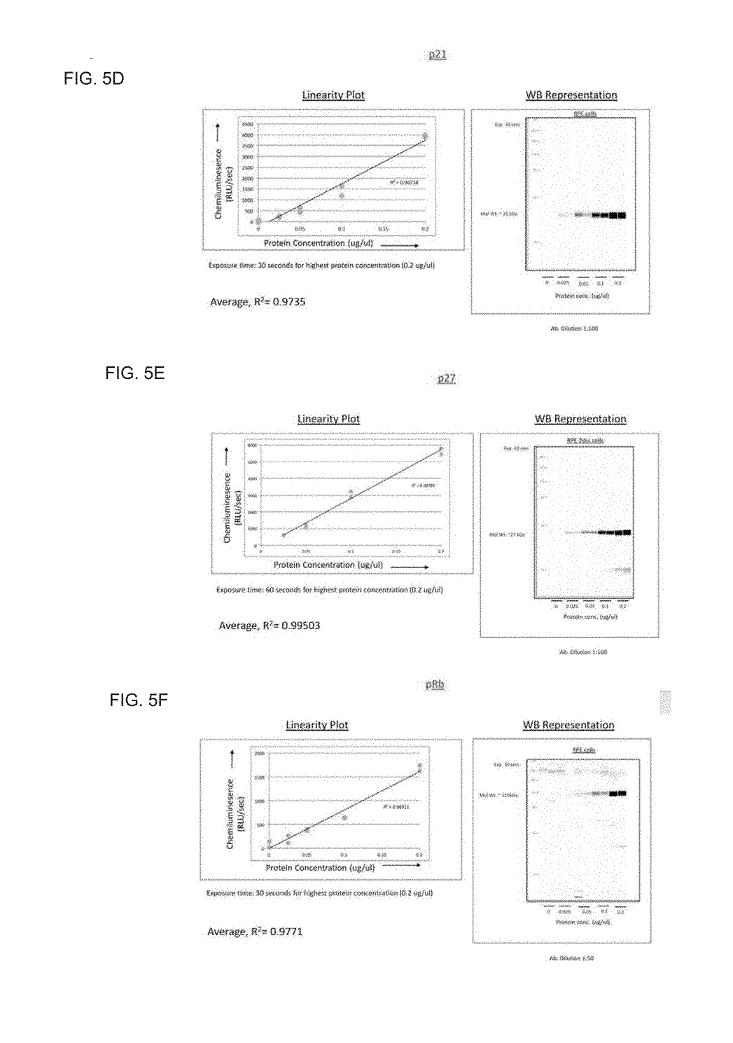

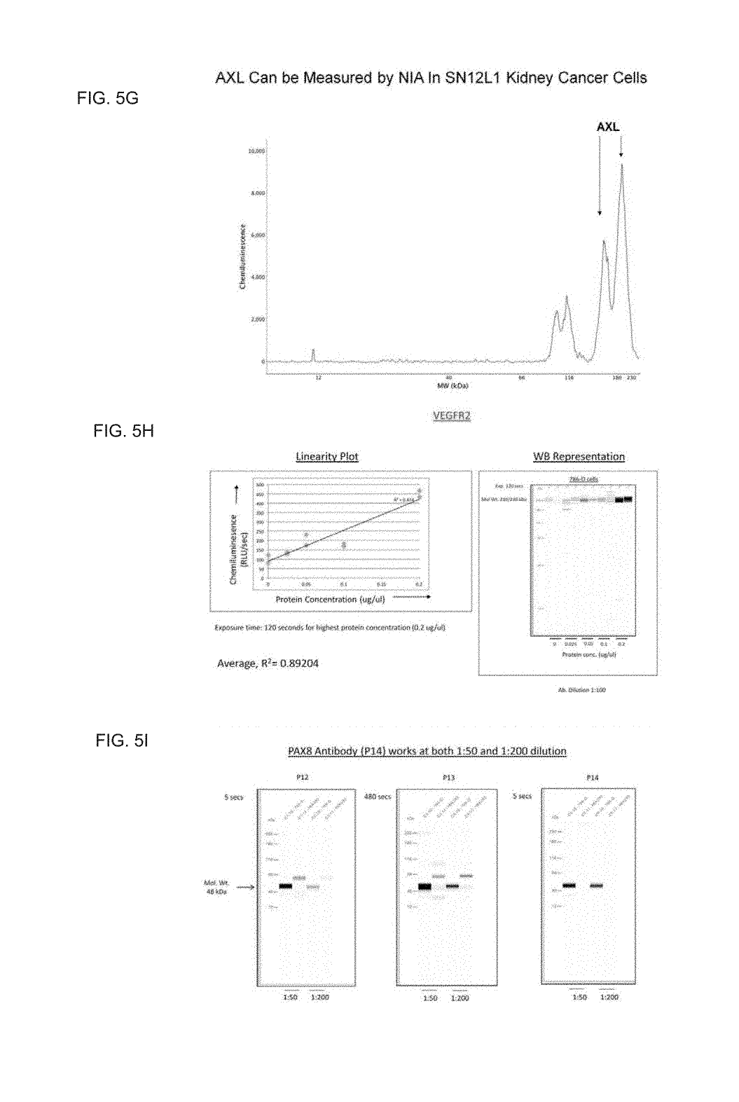

Shown in FIGS. 5A-5I are a series of NIA analyses for isoform distribution of human carbonic anhydrase 9, human alpha-tubulin (tubulin). In some conditions slices of tumor tissue from PDX mice were cultured in vitro and treated with either IQGAP1 WW domain peptide (WW), scrambled peptide (scr.; negative control for VVVV), trametinib (MEK inhibitor) or vehicle (DMSO; negative control for trametinib) for 2 days and the effect on tubulin was determined. Shown in C. is an analysis of human Cyclin D1; D. human p21; E. human p27; F. human retinoblastoma protein (pRb); G. human receptor tyrosine kinase AXL; H. human vascular endothelial growth factor receptor 2 (VEGFR2); I. human PAX8. These data show the applicability of NIA to a range of different proteins.

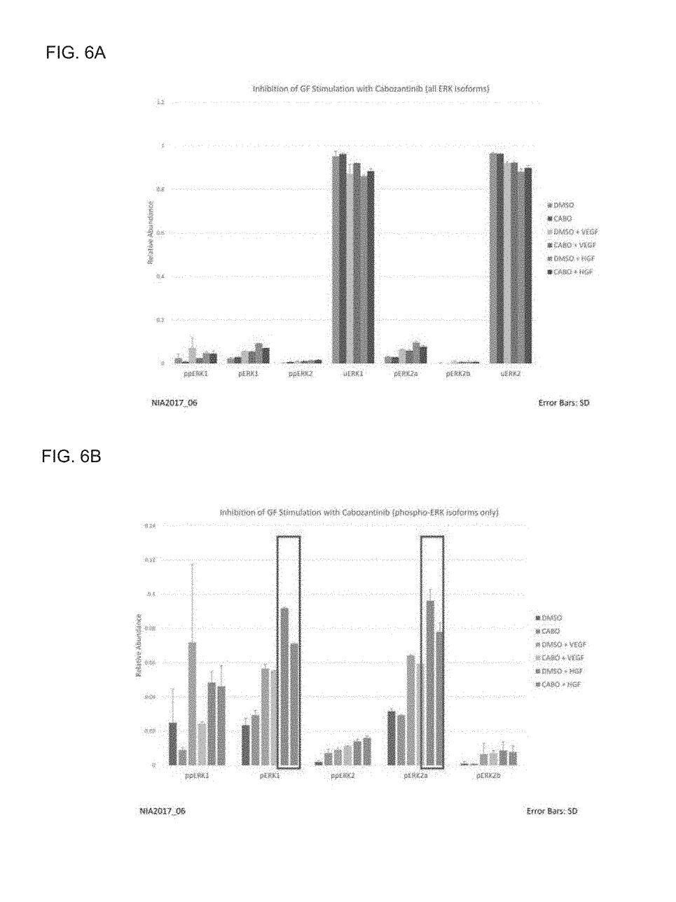

FIG. 6A illustrates quantification of relative abundance of ERK1 and ERK2 phospho-isoforms in cancer cells treated with cabozantinib (1 μM) or DMSO (negative control) for 1 day and stimulated with VEGF or HGF (10 ng/mL) for 10 min before cell lysis. The same data is presented in FIG. 6B but adjusted for scale without the most abundant isoforms. These data show that specific ERK isoforms have a distinct distribution pattern between treated and untreated samples.

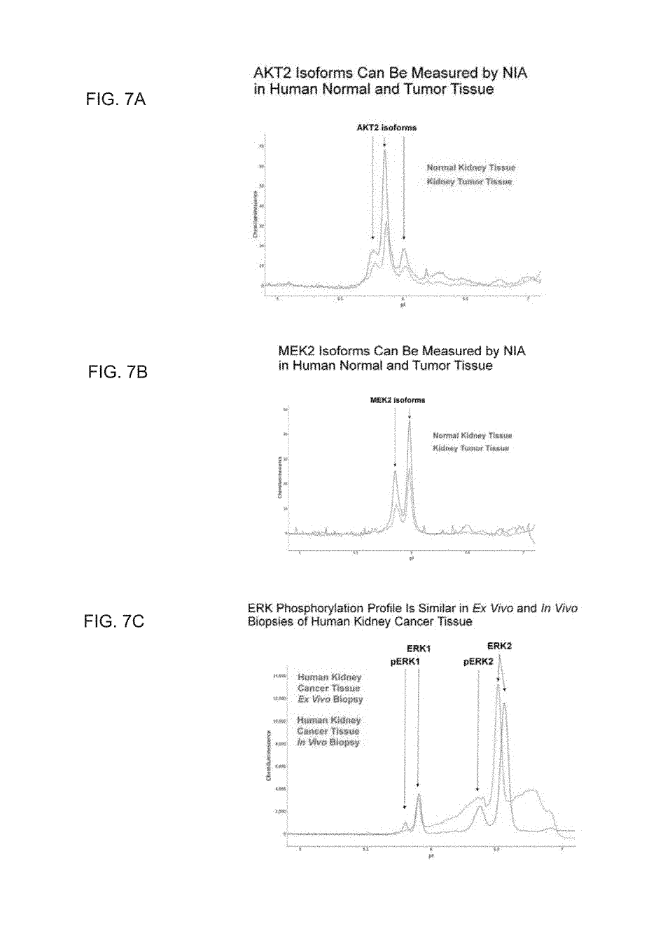

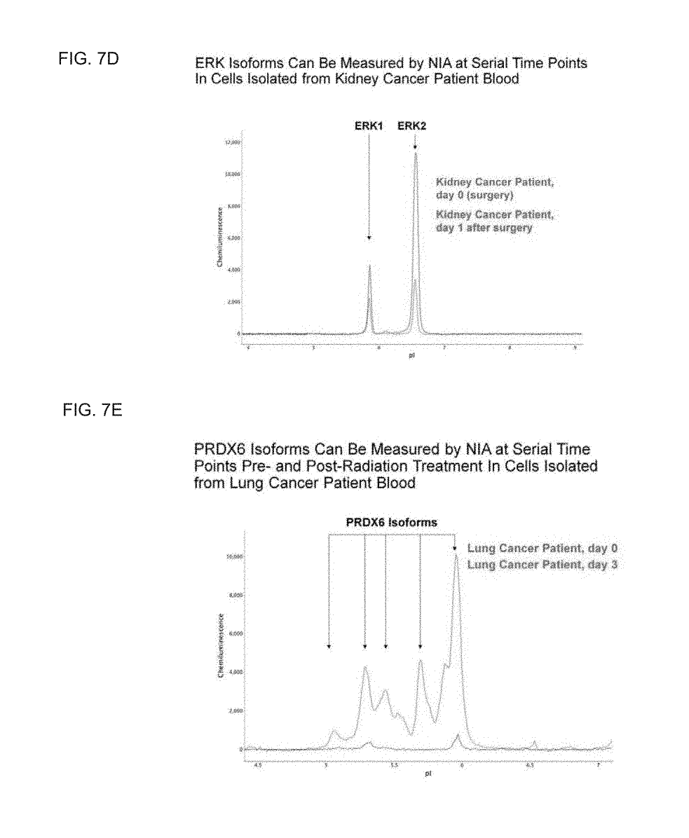

Shown in FIG. 7A is NIA analysis of AKT2 isoforms from unphosphorylated, mono- and multiple-phosphorylated AKT2 in cancer and matching normal tissue. In FIG. 7B, differently charged isoforms of MEK2 are identified, likely representing unphosphorylated, and phosphorylated MEK2. FIG. 7C and FIG. 7D illustrate ERK phosphorylation profiles, which is similar in both tissue samples with unphosphorylated ERK2 (ERK2) and mono-phosphorylated ERK2 (pERK2) being the dominant ERK isoforms. FIG. 7E shows analysis of multiple PRDX6 isoforms of different charges following radiation therapy, with profiles that differ pre- and post-treatment.

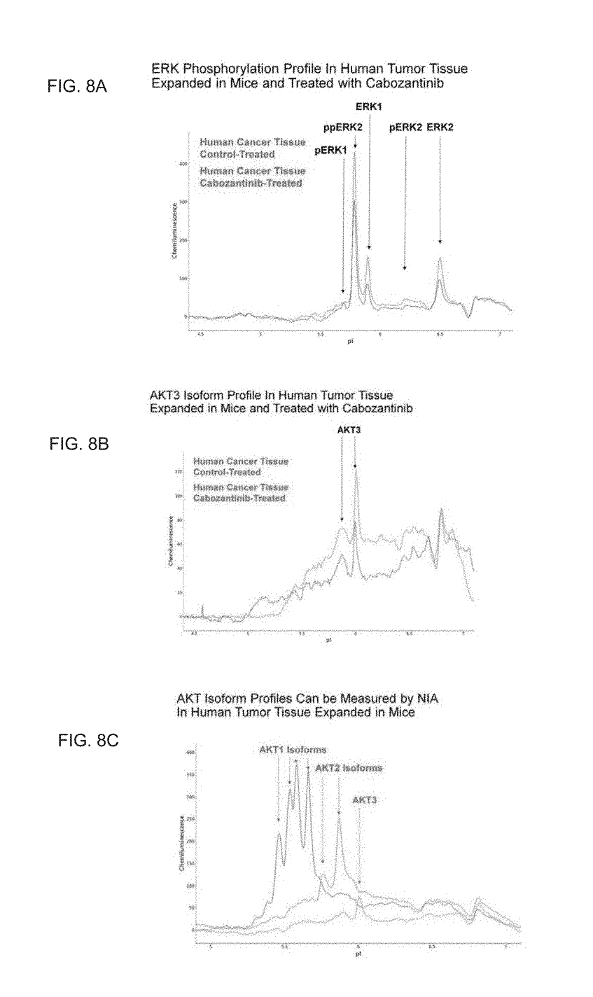

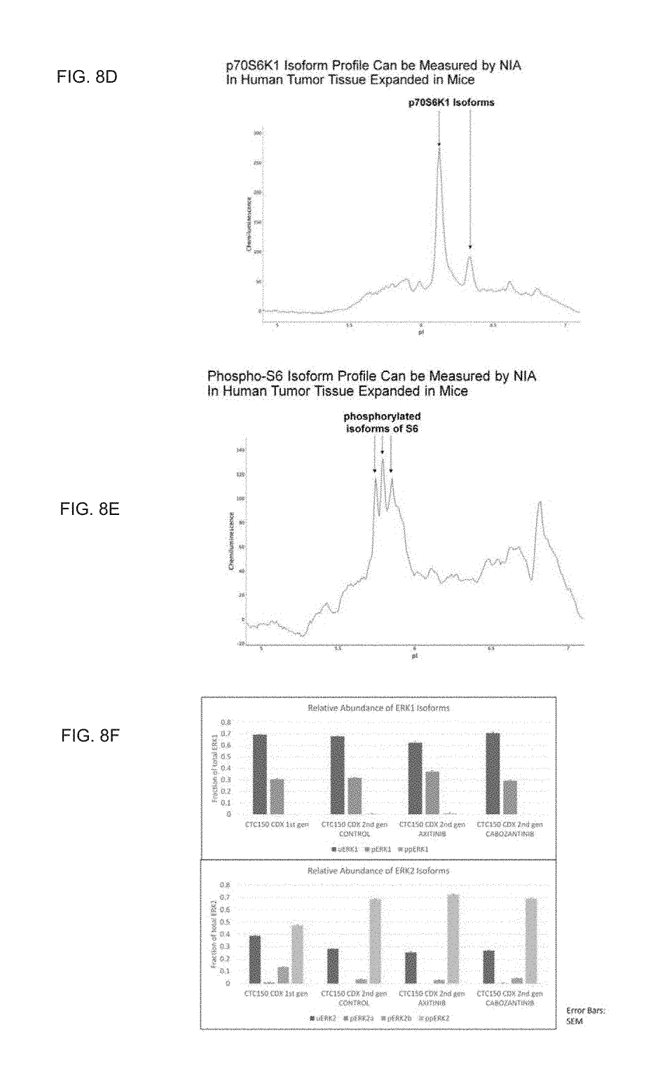

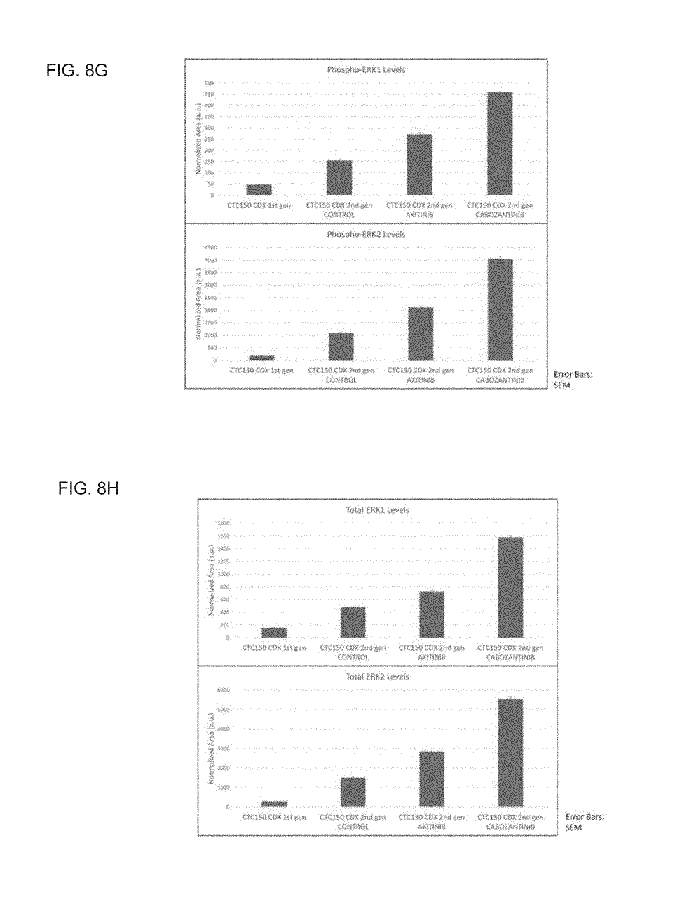

Shown in FIGS. 8A-8H are NIA analysis from lymphoma tissue expanded as a xenograft and treated with cabozantinib or vehicle (negative control) for 2 days. Tissue protein lysate was probed with an anti-pan-ERK antibody, anti-AKT3 antibody, antibodies specific to AKT1 identifying differentially charged (likely phosphorylated) isoforms of each AKT isoform; anti-p70S6K1 antibody; anti-phospho S6 antibody identifying three differently charged isoforms of phosphorylated S6 protein. Second-generation PDX mice were then treated with either cabozantinib, axitinib, or vehicle (negative control) for 2 days, then separated by NIA charge assay and probed with an anti-pan-ERK antibody. The relative abundance for each phospho-isoform of ERK1 and ERK2 was quantified as fraction of total ERK1 or ERK2, respectively. Quantitative ERK measurements (‘normalized area’) of the phosphorylated isoforms of ERK1 and ERK2 in human lymphoma expanded as patient-derived xenograft in mice were normalized to HSP70.

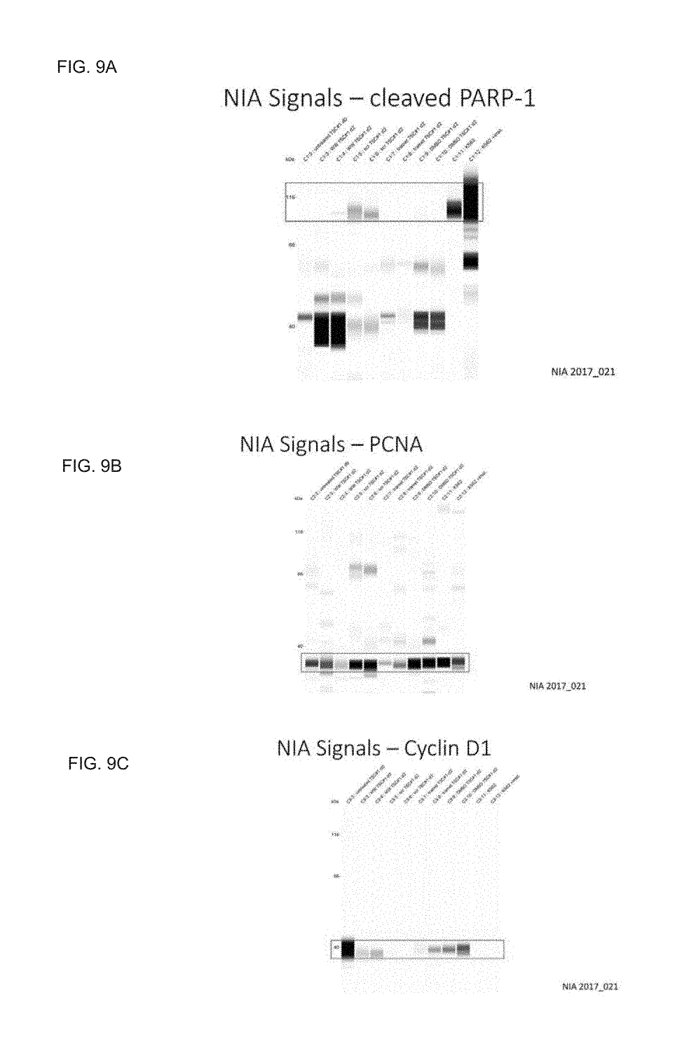

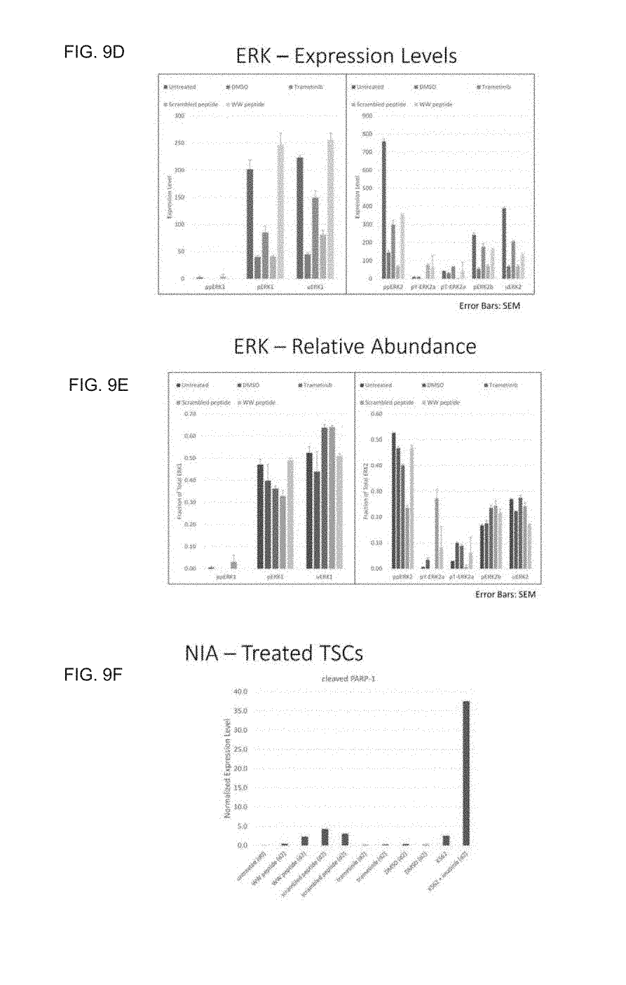

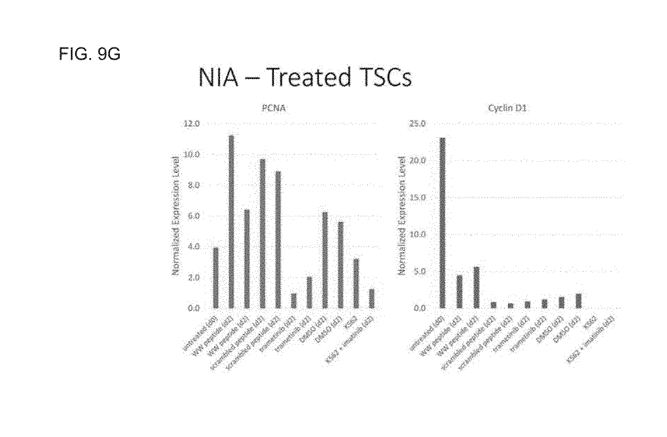

FIGS. 9A-9G illustrate detection of cleaved PARP-1 (apoptosis marker) by NIA size separation of protein lysates from tissue slice cultures (TSCs) from human kidney cancer tissue expanded in mice as patient-derived xenograft. Slices of tumor tissue from PDX mice were cultured in vitro and treated with either IQGAP1 WW domain peptide (WW), scrambled peptide (scr.; negative control for WW), trametinib (MEK inhibitor) or vehicle (DMSO; negative control for trametinib) for 2 days. Tissue protein lysate was probed with an anti-cl. PARP-1 antibody. Detection of PCNA, cyclin D1, ERK phospho-isoforms was also performed, quantified by normalization to HSP70.

These data show NIA can be used to measure a panel of apoptosis and proliferation markers, as well as signaling protein isoforms, in human cancer tissue, treated in vivo or ex vivo, to compare the response of individual samples to drugs of interest. Trametinib decreased cell cycle proliferation (PCNA) and did not change apoptosis (cleaved PARP-1), whereas WW peptide induced both apoptosis and increased cell cycle (PCNA).

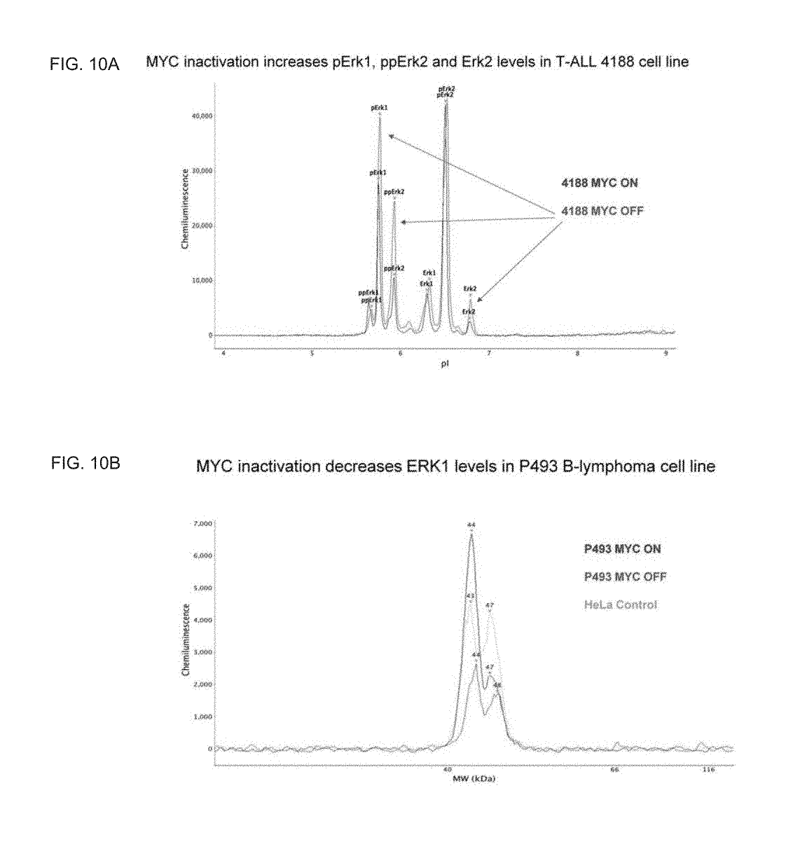

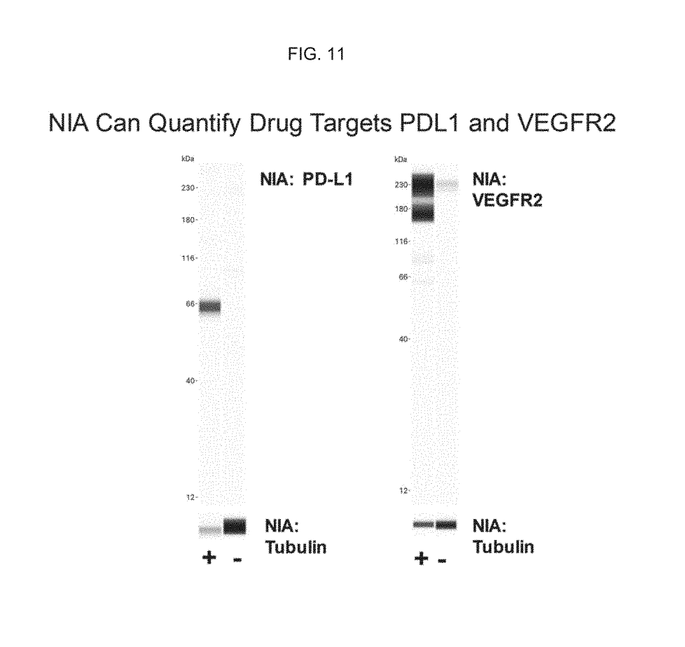

Shown in FIG. 10A and FIG. 10B, MYC inactivation increases pErk1, ppErk2, and Erk2 levels in T-ALL 4188 cell line. MYC inactivation decreases Erk1 level in Burkitt P493-6 cell line.

Shown in FIG. 11, NIA can be used to measure drug targets PDL1 and VEGFR2.

### Methods

Nano-Fluidic Proteomic Immunoassay (NIA).

The NIA experiments were performed using the Peggy Sue instrument (ProteinSimple, Inc.). Protein lysates from cells or tissues were prepared with 25 mM Bicine pH 7.6/0.6% CHAPS lysis buffer (ProteinSimple, Inc.) including a proprietary mix of protease and phosphatase inhibitors (Aqueous Inhibitor and DMSO Inhibitor, ProteinSimple, Inc.) according to the manufacturer's instructions. Protein lysates were then premixed with fluorescent standards of known isoelectric point (charge assay) or molecular weight (size assay) (ProteinSimple, Inc.) according to the manufacturer's instructions to a final concentration of 0.025-0.8 mg/mL. Briefly, for each capillary analysis, 400 nanoliters (for charge-separation assay) or 40 nanoliters (for size-separation assay) of protein lysate-fluorescent standard mix, corresponding to 8-24 ng of total protein, are automatically loaded into the microcapillary and electrophoretically separated by charge or size. After separation and photo-activated in-capillary immobilization, proteins were detected with antibodies and visualized by chemiluminescence.

NIA Peak Area Quantitation.

Quantitation of specific peaks is performed using Compass peak analysis software (ProteinSimple, Inc). The fitted peak and the fitted baseline (for background subtraction) were automatically selected by the software and manually confirmed and adjusted, if needed. The signal intensity was quantified by determining the area under each fitted peak.

NIA Pseudo-Blot Generation.

The pseudo-blots were created by linear mapping of the signal intensity to a grayscale image. Each pseudo-blot lane is representative of a single capillary and consists of horizontal bands corresponding proportionally to the signal intensity present at that position of the capillary. Absence of signal is white, while increasing signal is seen as an increasing dark band.

Human Tumor Samples.

Tissues were obtained from patients per Stanford University IRB-approved protocols and informed consent was obtained from all subjects. Tissue samples and cultured cells were pelleted, flash-frozen in liquid nitrogen, and stored at −80° C. For analysis, specimens were thawed on ice immediately before lysis.

Data and Statistical Analysis.

NIA Multipeak fitting and peak area calculations were done with Compass (Protein Simple), using Gaussian peaks with variable widths, as previously described. To obtain R² correlation coefficients, a linear fit was done in Excel (Microsoft, Inc.

Normalization of NIA Size Separation Data.

Normalization of NIA data was performed by quantifying tubulin signal in the same lysates as a loading control/“housekeeping” protein and dividing the measured peak area for protein of interest by the measured peak area of tubulin and expressed as a ratio. Across different experiments, a standardized lysate control was used for calibration of instruments and runs.

What is claimed is:

1. A method for monitoring response to cancer therapy, the method comprising;
treating cancer cells from an individual with a therapy of interest;
performing nanoimmunoassay (NIA) on a sample of the treated cancer cells;
determining a protein distribution pattern for a protein of interest;
comparing the protein distribution pattern to a reference protein distribution pattern to make a determination if the cancer cells are responsive to the therapy.

2. The method of claim 1, further comprising treating the individual in accordance with the determination of responsiveness.

3. The method of claim 1, wherein the cancer cells are treated in vivo.

4. The method of claim 1, wherein the cancer cells are a biopsy sample treated ex vivo.

5. The method of claim 1, wherein the sample of treated cancer calls is a blood sample.

6. The method of claim 1, wherein the sample of treated cancer calls is a blood sample.

7. The method of claim 1, wherein the sample of treated cancer cells is a fine needle aspirate.

8. The method of claim 1, wherein the reference protein distribution pattern is an untreated sample from the same individual.

9. The method of claim 1, wherein the sample is stored in cold medium for a period of from 4 to 48 hours.

10. The method of claim 9, wherein the sample is flash frozen after storage.

11. The method of claim 1, wherein the treatment comprises contacting cancer cells with a targeted therapeutic selected from cabozantinib, axitinib, trametinib, rigosertib, and IQGAP1 WW domain peptide.

12. The method of claim 1, wherein the treatment is radiation alone, or a combination with radiation.

13. The method of claim 11, wherein the protein of interest is selected from human glutaminase 1 (GLS1); human peroxiredoxin-6 (PRDX6); human carbonic anhydrase 9, human alpha-tubulin; human cyclin D1; human p21; human p27; human retinoblastoma protein (pRb); human receptor tyrosine kinase AXL; human vascular endothelial growth factor receptor 2 (VEGFR2); human PAX8; human PDL1.

14. The method of claim 11, wherein the NIA analyzes isoform distribution of a protein of interest selected from ERK, AKT1, AKT3, MEK2, PRDX6, p70S6K1, S6, PCNA, cyclin D1, cleaved PARP-1.
